(19) United States
(12) Patent Application Publication
(10) Pub. No.: US 2008/0286257 A1
(43) Pub. Date: Nov. 20, 2008

(54) Compounds for Use in Treating Abnormal Cell Proliferation, and Methods of Producing Same

(75) Inventors: Paolo Alberto Veronesi, Milan (IT); Pablo E.A. Rodriguez, Cordoba (AR)

Correspondence Address: STANDLEY LAW GROUP LLP, 495 METRO PLACE SOUTH, SUITE 210, DUBLIN, OH, 43017, US

(73) Assignee: THERAPICON Srl, Milan (IT)

(21) Appl. No.: 12/096,511

(22) PCT Filed: Sep. 29, 2006
(86) PCT No.: PCT/EP2006/066941

§ 371 Date: Jul. 17, 2008

(30) Foreign Application Priority Data
Dec. 6, 2005 (IT) MI2005A002327

(51) Int. Cl. A61K 38/47; C12N 9/36; A61P 35/00

(52) U.S. Cl. 424/94.61; 435/206

(57) ABSTRACT

The invention relates to the modification of recombinant human lysozyme from genetic engineering (rHLys) by reacting with activated polyethylene glycols (PEGs), which are partially binding or saturating with typical linkers one or more out of the six free amino groups of the five lysines (K) present in the rHLys chain. The resulting conjugate compounds of the invention (PEG-rHLys) and their addition salts, inhibit the abnormal cell proliferation and show remarkable antiproliferative and antimetastatic effects. The invention relates also to the use of the new compounds in the preparation of pharmaceutical compositions for treating a disease associated with abnormal cell proliferation, including cancer, by administering said conjugates to a subject in need thereof.

The present invention relates generally to the modification of recombinant human lysozyme from genetic engineering (rHLys) by reacting with activated polyethylene glycols (PEGs), which are partially binding or saturating with typical linkers one or more out of the six free amino groups of the five lysines (K) present in the rHLys chain.

The resulting conjugate compounds of the invention (PEG-rHLys) and their addition salts, reduce the abnormal cell proliferation and show remarkable antiproliferative and antimetastatic effects. The invention relates also to the use of the new compounds of the invention in the preparation of pharmaceutical compositions for treating a disease associated with abnormal cell proliferation, including cancer, by administering said conjugates to a subject in need thereof.

### 1.0 FIELD OF THE INVENTION

Although the incidence of cancer has increased, and cancer is a leading cause of death in developed countries (it is responsible for one out of five deaths, and it affects all age groups, sexes and ethnic groups), the number of cancer deaths has slightly declined in recent years in developed countries. This is due in part to better therapy designs but also to prevention programs and better detection of some cancers at an earlier stage.

Nowadays an increasing number of scientific publications indicate that the abnormal cell proliferation, including cancer, is an expression of disorders affecting the immunological system in mammalians. Therefore the use of the traditional chemiotoxic therapies, widely and increasingly used during the past decades and also at the present, affecting both the abnormally proliferating cells but also the healthy cells of the immunological system, is now strongly questioned and in future it will be probably subject to a radical revision. However, in spite of the achievements in treatment and prevention of cancers, several improvements are still awaited:
effective therapies to modify the disorders causing early stage cancer; • effective treatments to prevent proliferation and reduce relapses without major and irreversible damages to healthy cells of the immunological system;
alternative immunological therapies for preventing the metastatic proliferation;
alternative therapies for treating tumours refractory to the standard therapies, which are generally rather invasive and toxic.

Therefore, modifiers of the immunological system such as lysozymes could possibly represent such a new alternative therapy to advantageously meet the above needs, either when used alone or in combination with the standard chemotoxic drugs.

Lysozymes are enzymes from peptidic origin, present in the animal and vegetal world. Their structures are species-related and, despite significant differences in terms of homology, their enzymatic mechanism is comparable. However, lysozymes are widely distributed in the mammalian body with different and often unknown functions. They are present in variable concentrations in saliva, in tears, in milk, in leukocytes, in cervical mucus. In 1922 Alexander Fleming accidentally discovered that a substance present in his own nasal mucus was able to lysate some bacterial strains. This substance was later identified as natural human lysozyme. In spite of several scientific publications on lysozymes, the potential medicinal use of human lysozyme has been completely neglected in view of the unclear pharmacological properties and vague indications. In fact, there are no clear indications of any specific medicinal properties. Moreover, there are objective difficulties to extract natural human lysozyme from human organic materials and to make it available in sufficient amounts.

Most of the scientific publications report that natural human lysozyme elicits a direct action to degrade the link β-(1->4) between N-acetylmuramic acid and N-acetylglucosamine of peptidoglycans present in the wall of the cell membrane of bacteria, thus determining the lysis of the same. In this connection, Gram positive bacteria are generally more sensitive to natural human lysozyme action than Gram negative, probably because the latter ones present the external membrane as a further barrier. This direct action of natural human lysozyme is important for the natural prevention or therapy of some bacterial diseases, such as faringitis, congiuntivitis, otitis, sinusitis, adenitis, uretritis, vaginitis, cistitis, substantially caused by microorganisms of S.

The second main activity that other scientists attribute to natural human lysozyme is that the mucopeptidic fragments or debris of the cell membranes produced from the lysis of the bacteria could possibly elicit a sort of indirect stimulation of the immunological system to produce antibodies against such bacteria.

By contrast, healthy human cells are not destroyed by lysozyme, so that the possibility of toxic effect on the different human tissues and organs is rather remote. However, like other similar peptides, lysozyme is either inactivated when exerting its specific biological functions or it is degraded by other proteolytic enzymes, both factors which reduce its natural half-life.

Nevertheless, considerable steps forward in this field have been made during these most recent years. In fact, U.S. Pat. No. 6,642,437 discloses (1) a method for producing proteins in plant seeds, while Huang J. et al. (Molecular Breeding, 10: 83-94, 2002) explored more specifically (2) the expression of human lysozyme in maturing rice grains transformed using the codon-optimized structural gene for human lysozyme. Biochemical, biophysical and functional comparisons of native and recombinant human lysozyme revealed an almost identical N-terminal sequence, molecular weight, and specific activity on bacteria.

Furthermore, similar bactericidal activity was displayed towards a laboratory strain of Escherichia coli. The aim of the above studies was basically to yield improved nutritional quality of infant formulas and baby foods with rice flour or rice extract containing recombinant human lysozyme. Further publications disclose: (3) methods and compositions for high level expression of heterologous polypeptides in the grains of transgenic plants, from which they are extracted in a substantially impure form and low concentrations and administered in a vehicle comprising a processed food in which the extract is mixed (WO 02/064750); (4) food and food additives comprising one or more milk proteins produced in the seeds of a transgenic plant and method of making the same, with the aim to obtain improved infant formula comprising such food supplement composition (WO 01/83792; WO 02/064814); (5) a mature, transgenic monocot seed that yields, by extracting ground seeds with an aqueous medium, a total soluble protein fraction containing at least 3.0% by total protein weights of a human milk protein, selected from the group consisting of lactoferrin, lysozyme, lactoferricin, lactadherin, kappa-casein, haptocorrin, lactoperoxidase, alpha-lactalbumin, beta-lactoglobulin, alpha-casein, beta-casein and alpha-1-antitrypsin; and a milk-protein composition comprising a total soluble protein fraction in the dried form for use as an additive for an ingestible food or feed (WO 05/017168).

In addition to the above references, a recombinant human lysozyme, typically characterized as the natural human lysozyme for containing six accessible free amino groups of the five lysines in the peptidic chain, may be also obtained by genetically-modified host cells, already known in the art, such as prokaryotic microorganisms like Escherichia coli or Bacillus subtilis or eukaryotic cells like yeast or mammalian cells. Methods for producing said recombinant human lysozyme are described in EP1111054 (U.S. Pat. No. 6,660,512), EP1111058 (U.S. Pat. No. 6,528,297), EP1111059 (U.S. Pat. No. 6,436,688) and EP 1111060 (U.S. Pat. No. 6,743,617). Therefore, methods are now known in the art for producing and isolating recombinant human lysozyme, using different techniques, but its practical application is substantially limited to the nutritional field.

In view of the availability of larger quantities of recombinant human lysozyme at certain concentrations, the authors decided to explore the possibility to substantially modify its original chemical structure to yield derivatives almost devoid of its typical proteolytic antimicrobial activity on bacteria and with an increased resistance to the proteolytic degradation.

In this connection the authors formulated the theory that the elimination of the above disadvantages in the new derivatives could unexpectedly lead to unforeseen medicinal properties and applications, such as the modification of the course of disorders related to a humoral defence reduction, which seems to be a cause of abnormal cell proliferation in tumour and metastasis.

### 2.0 GENERAL

Surprisingly, this target has been achieved by the authors of the present specification by substantially modifying the recombinant human lysozyme (rHLys) obtained from genetic engineering.

In fact, the modified derivatives of rHLys, despite being deprived of the proteolytic antimicrobial effect typically expressed by the natural or recombinant human lysozyme, showed reduced immunogenicity and remarkable resistance to the proteolytic degradation, but also unexpectedly elicited unforeseen antitumour and antimetastatic effects on abnormal cell proliferation processes.

Therefore the main objective of the present invention is to provide conjugates of recombinant human lysozyme (rHLys) from genetic engineering with activated polyethylene glycols (PEGs), which are binding with one or more out of the six free amino groups of the five lysines present in rHLys chain.

In addition, the compounds of the invention are characterized not only from a different degree of pegylation, but also from the variable molecular weight and type of linkage, being both the typical expression of the activated PEGs, which is used for the condensation reaction.

In a second embodiment the invention relates to the synthesis methods to produce and purify said compounds, in order to achieve a standard pharmaceutical quality. Accordingly, the first step is to react rHLys with different quantities of a selected activated PEG where a terminal group, typically a hydroxy or amino group, has been replaced by an activated linker. The kind of linkage between one or more out of the six free amino groups of rHLys with an activated PEG is also a feature of this invention and typically depends on the type and quantity of the activated PEG used to yield mono-, di-, tri-, tetra-, penta- or hexa-pegylated-recombinant human lysozyme.

The present invention also relates to pharmaceutical compositions comprising at least one compound as defined above in a pharmaceutically acceptable carrier or support, optionally in association with another active ingredient.

In another embodiment, the invention also relates to the use of a compound, as defined above, for the manufacture of a medicament for the treatment in human subjects of diseases associated with abnormal cell proliferation and migration, such as cancer and metastasis.

In fact, as it will be further disclosed, a compound of present invention elicits a significant inhibition on abnormal cell proliferation and metastasis experimentally induced in animal models. Said compound is also remarkably effective at reducing or arresting growth of abnormal cell proliferation, such as primary tumour and metastasis.

### 3.0 DESCRIPTION OF PREFERRED EMBODIMENTS

Accordingly, one aspect of the invention is to provide a compound having the general formula (I):

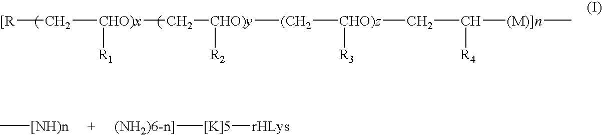

wherein:
R is a lower alkoxy, more preferably a methoxy group;
R₁, R₂ and R₃, independently from each other, are selected from a hydrogen atom or a lower alkyl group;
R₄ is a hydrogen atom or a lower alkyl group, with the proviso that R₄ is absent when M is a hydrogen atom;
x, y and z are selected from any combination of numbers such that the resulting molecular weight (MW) of any of the selected polyethylene glycol moieties vary from 300 to 66,000 Daltons (from 0.3 to 66 Kilodaltons, i.e. from 0.3 KDa to 66 KDa);
M is a linker group;
n represents an integer from 1 to 6, being the number of polyethylene glycol moieties linking one or more out of the six free amino groups of the five lysines present in rHLys;
[(NH)n+(NH₂)6−n] are the six amino groups of the five lysines present in rHLys, where (NH)n represents one or more amino groups linked to “n” polyethylene glycol moieties and (NH₂) 6−n represents one or more residual free amino groups;
[K]5 are the five lysines (K) present in rHLys chain; and
[(NH)n+(NH₂)6−n]-[K]5-rHLys is a detailed representation of recombinant human lysozyme where the five lysines [K]5 present in recombinant human lysozyme and the six linked or free amino groups [(NH)n+(NH₂)6−n] are drawn separately.

Preferably the linker group M is derived from one or more compounds that react with a linear or branched polyethylene glycol to produce an activated linear or branched polyethylene glycol, which in turn will react and substitute one hydrogen of one or more free amino groups of the lysines in the recombinant human lysozyme, and thereby link together the polyethylene glycol moiety and the recombinant human lysozyme.

Further preferably the linker group M is selected from one or more of linkers (a), (b), (c) or (d), each originating a different type of linkage, where:
(a) is a hydrogen atom, with the proviso that R₄ is absent (type of linkage: amine);
(b) is a methylene group (type of linkage: amine but with a methylene added to the polyethylene glycol);
(c) is

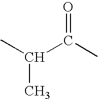

(type of linkage: amide); and
(d) is

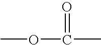

(type of linkage: urethane).

The present invention includes any and all tautomers, optical and geometrical isomers, racemates, salts, hydrates, solvates and mixtures thereof, of the compound having the general formula (I).

It is intended that stereoisomers (optical isomers), as separated, pure or partially purified stereoisomers or racemic mixtures thereof, of the compound having the general formula (I) are also included in the scope of the invention.

In accordance with this invention R₁, R₂ and R₃ can be any lower alkyl. The term lower alkyl designates lower alkyl groups containing from 1 through 6 carbon atoms, such as methyl, ethyl, n-propyl, isopropyl, and so on, but preferably a methyl group.

Moreover R can be a lower alkoxy, where the term lower alkoxy designates lower alkoxy groups containing from 1 through 6 carbon atoms, such as methoxy, ethoxy and so on, but preferably a methoxy group.

More preferably R₁, R₂ and R₃ can be simultaneously a hydrogen atom. In a second aspect of the invention, the compounds of the invention are synthesized by a chemical condensation reaction of rHLys with an activated PEG, where a terminal group, preferably a hydroxy or amino group, opposite to R (alkoxy group, preferably a methoxy group), has been replaced by an activated linker. Therefore the activated reagent reacts either with one or more out of the six accessible free amino groups of the five lysines [K]5 present in rHLys. The preferred activated PEG, used to yield the compound of general formula (I), is typically an activated polyethylene glycol having a general formula selected from (II)-(A), (III)-(B), (IV)-(C) or (V)-(D), which are drawn below, where the polyethylene glycol moiety:

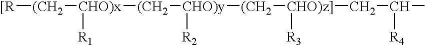

is concisely represented, only for drawing purposes, as:

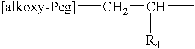

In addition, the moiety: [alkoxy-Peg]-CH₂—CHR₄-activating group where alkoxy is methoxy and R₄ is a hydrogen atom, is represented as m-PEG-activating group. Some of the preferred are reported hereby:

### Preferred Activated PEGs (General Formulae and Nomenclatures)

### (A)• TalkoxyPEG (Contracted Name)

alkoxypolyethylene glycol tresylate (chemical name)

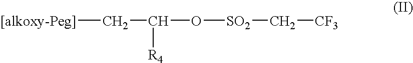

In the most preferred TalkoxyPEG, alkoxy (R) is a methoxy and R₄ is a hydrogen atom: TmPEG (methoxypolyethylene glycol tresylate). The activated reagent (A) [formula (II)] forms an aminic linkage when reacted with rHLys.
   (B)•alkoxyPEG-ButyrALD (Contracted Name)
alkoxypolyethylene glycol butyraldehyde (chemical name)

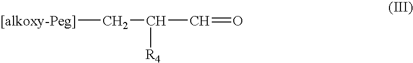

The most preferred alkoxyPEG-ButyrALD is that where alkoxy (R) is a methoxy and R₄ is a hydrogen atom:
mPEG-ButyrALD (methoxypolyethylene glycol butyraldehyde). The activated reagent (B) [formula (III)] forms a methylene aminic linkage (a methylene group is added to the polyethylene glycol moiety) when reacted with rHLys.
   (C)• alkoxyPEG-SMB (Contracted Name)
alkoxypolyethylene glycol succinimidyl alfa-methylbutanoate (chemical name)

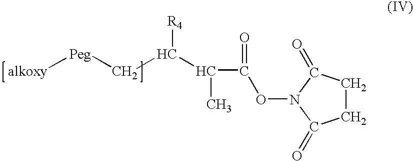

The most preferred alkoxyPEG-SMB is that where alkoxy (R) is a methoxy and R₄ is a hydrogen atom: mPEG-SMB (methoxypolyethylene glycol succinimidyl alfa-methylbutanoate). The activated reagent (C) [formula (IV)] forms an amidic linkage when reacted with rHLys;
   (D)• alkoxyPEG-OSu or alkoxyPEG-SC (Contracted Names)
alkoxypolyethylene glycol succinimidyl carbonate (chemical name)

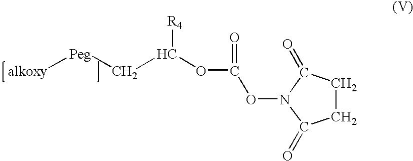

The most preferred alkoxyPEG-OSu is that where alkoxy (R) is a methoxy and R₄ is a hydrogen atom:
mPEG-OSu (methoxypolyethylene glycol succinimidyl carbonate). The activated reagent (D)) [(formula (V)] forms an urethanic linkage when reacted with rHLys.
   Wherein, in the above chemical formulae:
R, R1, R2, R3 and R4 are as in the general formula (I);
x, y and z are selected from any combination of numbers such that the resulting molecular weight (MW) of any of the selected polyethylene glycol moieties vary from 300 to 66,000 Daltons (from 0.3 to 66 Kilodaltons, i.e. from 0.3 KDa to 66 KDa);
with the proviso that:
R is preferably a methoxy group;
R₄ is a hydrogen atom, but absent when M is a hydrogen atom.

Another aspect of the present invention is that, by using an activated PEG reagent of formula (II), (III), (IV) or (V) to produce the conjugates as defined above, it is possible to form a different type of linkage (M) between one or more out of the six free amino groups of the five lysines [K]5 present in the rHLys chain.

In a further aspect of the invention, the different degree of pegylation formed in the conjugates as above described through the use of different quantities or of an excess of any one of the activated PEG of formulae (II), (III), (IV) or (V), is also a feature of this invention.

In fact, “n” represents an integer from 1 to 6 in the general formula (I), being the number of the polyethylene glycols linked to the rHLys chain. Since 6 is the total number of free amino groups of the 5 lysines [K]5 present in the rHLys chain, which are potentially accessible to the activated reagent, “n” represents also the equimolar number of each used activated reagent, which may therefore vary from 1 to 6.

Despite “n” being an integer varying from 1 to 6, a preferred embodiment of the instant invention is a conjugate where “n” is comprised from 3 to 6. Such a preferred compound is produced by conditions that a high yield is obtained of the desired rHLys conjugate, where from 3 to 6 amino groups have reacted with one of the selected activated reagent of formula (II), (III), (IV) or (V).

In another different aspect of this invention, the higher the value of x, y and z, the higher the molecular weight (MW) of the activated reagent and of the resulting conjugate.

In accordance with a preferred embodiment of this invention x, y and z are any number so that the polyethylene glycol of the activated reagent which forms the conjugate with the free amino group of lysines [K]5 has an average molecular weight varying from 300 to 66,000 Daltons, preferably from 400 to 10,000 Daltons, typically from 1,000 to 6,000 Daltons (from 1 to 6 Kilodaltons, i.e. from 1 KDa to 6 KDa).

When a compound according to the invention is in a form of salt, it is preferably a pharmaceutically acceptable salt. Such salts include pharmaceutically acceptable acid addition salts. Acid addition salts include salts of inorganic acids as well as organic acids. Representative samples of suitable inorganic acids include hydrochloric, hydrobromic, hydroiodic, phosphoric, sulfuric, nitric acids and the like. Representative samples of suitable organic acids include formic, acetic, trichloroacetic, trifluoroacetic, propionic, benzoic, cinnamic, citric, fumaric, glycolic, lactic, maleic, malic, mandelic, oxalic, picric, pyruvic, succinic, methanesulfonic, ethanesulfonic, tartaric, ascorbic, pamoic, gluconic, citraconic, aspartic, stearic, palmitic, EDTA, glutamic, benzenesulfonic, p-toluenesulfonic acids and the like. Further examples of pharmaceutically acceptable inorganic or organic acids addition salts include the pharmaceutically acceptable salts listed in J. Pharm. Sci., 66, 2, 1977. The pharmaceutically acceptable salts are prepared by reacting the compound of formula (I) with 1 up to 6 equivalents of an acid. A suitable solvent or a mixture thereof may be used.

The resulting salt is then precipitated, separated by filtration and dried, or lyophilized according to one of the several methods well known to the skilled artisan.

When the activated reagent of any one of formula (II), (III), (IV) or (V) is reacted with rHLys, which contains six accessible free amino groups, a conjugate may be produced also as a mixture of various reaction products of rHLys with the polyethylene glycol. This is signified by “n” in the general formula (I). For example, since rHLys contains only 6 accessible free amino groups of the 5 lysines [K]5, the activated reagent can partially react with one or with two or three or more out of the six free amino groups.

This depends on the quantities of activated reagent that are used in the reaction mixture. If an excess of activated reagent is used, the saturation of all six free amino groups of rHLys is achieved, thus yielding an hexapegylated conjugate of rHLys. In the situation that a lower stoichiometric quantity of activated reagent is used the mixture contains partially conjugated reaction products. Since the various conjugated reaction products in this mixture may present vastly different molecular weights, depending on the values of “n”, such as 1, 2, 3 or 4 and so on, these reaction products can be separated by a conventional method such as chromatography in a column.

In a most preferred embodiment of the invention “n” is 6 and this represents the complete saturation of all six accessible free amino groups of the five lysines [K]5 present in rHLys. This condition may be obtained when an appreciable excess of activated reagent is used.

In the case of incomplete pegylation of the six accessible and free amino groups of [K]5, represented as [(NH)n+(NH₂)6−n] in the general formula (I), the partially reacted conjugate may be obtained by gradual steps, where the activated reagent will firstly react with one and then with two and progressively with one more of the remaining accessible free amino groups of [K]5 present in rHLys.

In fact, by regulating the concentration of the reagents, such as rHLys and the activated reagent, and the reaction conditions of the condensation, one can regulate the degree of pegylation of the amino groups contained in the lysines of the enzyme. In fact the condensation can be regulated within a certain extent either by using an excess of activated reagent or by allowing the condensation reaction to proceed longer or by utilising other different or stronger conditions.

To produce a conjugate of general formula (I), where preferably alkoxy is a methoxy (—OCH₃), each of the following reaction schemes may be used:

### Synthesis Schemes

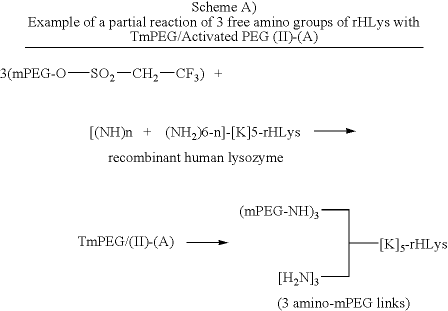

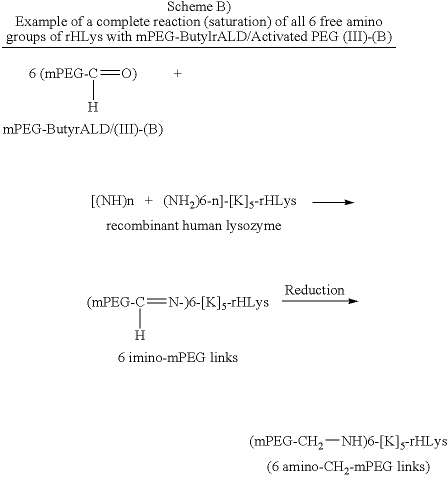

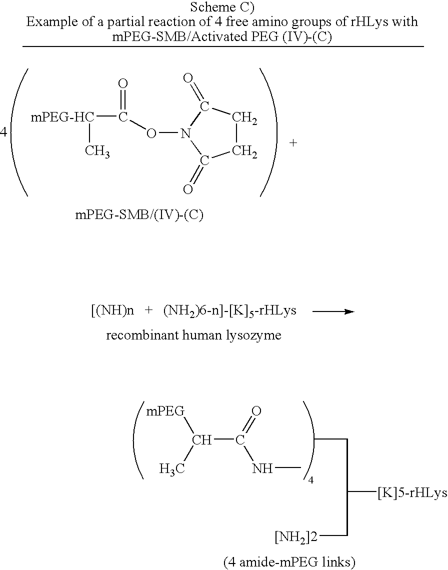

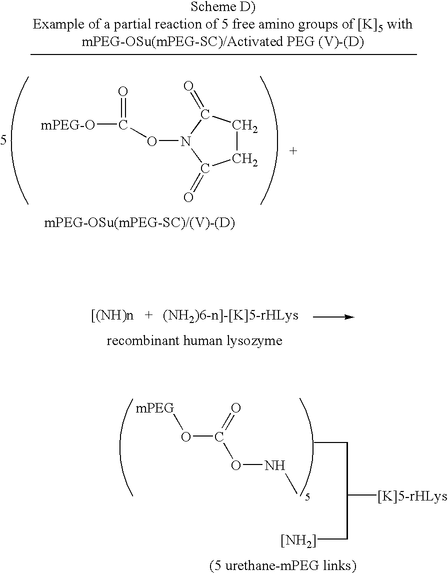

The advantage to use a specific activated reagent lies in an enhancement of the yield of the desired conjugate, which can depend individually or globally on the above mentioned variable reaction conditions, including the molecular weight of the activated reagent.

Generally the reaction may produce in any case a mixture of pegylated enzyme conjugates of various molecular weights depending upon the number of free amino groups within the enzyme which have been linked to the activated reagent and upon the different reaction conditions, as described above.

The PEG-rHLys conjugates may be then separated into their individual components and purified by conventional methods such as high performance liquid chromatography (HPLC), gel electrophoresis, molecular ultrafiltration, or better column chromatography, as it will be better indicated in the specific examples. Any conventional equipment and conditions for separating compounds by molecular weight with high performance liquid chromatography, gel electrophoresis, molecular ultrafiltration and column chromatography may be used to separate the conjugates.

The methods as described above for preparing compounds of formula (I) represent a further embodiments of the present application. It should be understood that other ways of producing these compounds may be designed by the skilled person, based on common general knowledge and following guidance disclosed in this invention.

Similarly, a conjugate of general formula (I), prepared by a condensation reaction in the same fashion an activated polyethylene glycol reagent with recombinant human lysozyme, commercially available from genetically-modified host cells containing a particular gene construction to yield said recombinant human lysozyme by means of techniques known to the skilled person, is also a feature of the instant invention. Said recombinant human lysozyme obtained from host cells shall also present, as the natural one, six accessible free amino groups in the five lysines of the peptidic chain.

In fact it should be understood also that recombinant human lysozyme from host cells may be conveniently modified by the skilled person by using the described activated polyethylene glycol reagents, reaction schemes and conditions, simply following the guidance disclosed in this invention.

In another embodiment of the instant invention the authors have surprisingly found that the PEG-rHLys conjugates show biological and pharmacological effects substantially different from the original rHLys from which it is derived.

In fact the above conjugates are almost devoid of the original proteolytic activity on bacteria, but are not readily susceptible to in vivo hydrolytic and enzymatic cleavages and therefore are less subject to the disadvantages of a rapid degradation present in the original rHLys.

Furthermore the authors have surprisingly found that PEG-rHLys conjugates are very useful in preventing and reducing the abnormal cell proliferation, causing the most common oncological pathologies. In fact the authors have surprisingly found that PEG-rHLys conjugates remarkably prevent the onset, the growth and the matastatic process of a tumour experimental model in laboratory animals.

A further object of this invention relates to a pharmaceutical composition comprising at least one compound of formula (I), as defined above, and a pharmaceutically acceptable vehicle or support. The compounds of the invention may be formulated in various forms, including solid and liquid forms, such as tablet, capsule, pill, dragee, granule, powder, syrup, suspension, emulsion, solution, lyophilized powder, and the like.

The compositions of the invention may contain pharmaceutically acceptable diluents, fillers, lubricants, excipients, solvents, binders, stabilizers, thickening agents, and the like. Diluents that may be used in the compositions of the compounds of the invention include but are not limited to dicalcium phosphate, calcium sulphate, lactose, microcrystalline cellulose, kaolin, mannitol, sucrose, dextrose, sodium chloride, starch and for prolonged release tablet the hydroxypropyl methylcellulose (HPMC), shellac, and the like. The binders that may be used in the compositions for wet granulation include but are not limited to starch, gelatin, natural and synthetic gums, povidone, sodium alginate, polyethylene glycols, and the like and for direct compression include but are not limited to microcrystalline cellulose, spray dried and anhydrous lactose, starch, dicalcium phosphate, and the like. The fillers such as sucrose, glucose, dextrose and lactose, and the like may be also used. Natural and synthetic gums that may be used in the compositions include but are not limited to sodium alginate, veegum, carboxymethyl cellulose, methyl cellulose, povidone.

Stabilizers that may be used include but are not limited to polysaccharides such as acacia, agar, alginic acid, guar gum and tragacanth, and disintegrants include croscarmellose sodium type A and other starches and cellulose derivatives water soluble, and the like.

Lubricants, glidants and antiadherents that may be used include but are not limited to metallic stearates (magnesium, calcium and zinc), stearic acid, hydrogenated vegetable oils, talc, corn starch, polyethylene glycols, microfine silicas, and the like. Surfactants like sodium lauryl sulfate and dioctyl sodium sulfosuccinate (DDS) may be also used.

Solvents that may be used include but are not limited to Ringer's solution, water, bidistilled water, propylene glycol (alone or in water), phosphate buffered saline, balanced salt solution, and other conventional and pharmaceutically acceptable fluids.

The compounds of formula (I) can be used in a substantially similar manner to other known anti-tumour agents for treating various pathological conditions. For the compounds of the invention, the dose to be administered, the dosage frequency, the total daily dose, the length of treatment can significantly vary for each subject with the specific potency of the compound, the chosen route of administration, the type of tumour, the age and the body weight of the recipient, the rate of excretion, drug combination, and the general conditions of the patient undergoing therapy.

Accordingly, the dosage to be administered is not subject to definite bounds, but it will usually be an effective amount to achieve the desired pharmacological and therapeutic effects.

Accordingly, optimal therapeutic concentrations will be best determined and adjusted through routine clinical experimentation in patients suffering from different diseases to be treated.

In fact, an oncologist skilled in the art of cancer treatment will be able to ascertain, without undue experimentation, appropriate protocols for the effective administration of the compounds of the present invention, such as by referring to the earlier published studies on compounds found to have anti-tumour properties, and then to adjust the dosage regimen in relation to the achieved therapeutic results.

Nevertheless the following dosage and regimen are tentatively reported hereby as a non-binding guidance.

The dosages and the dosage regimen in which the compounds of formula (I) are administered will vary according to the dosage form, mode of administration, the condition being treated and particulars of the subject being treated, and the like.

The compounds according the present invention may be preferably used orally. The compounds are suitably administered at the rate of 10 mg to 200 mg per day per Kg body weight, more preferably from 20 mg to 100 mg per day per Kg of body weight.

The required daily dose can be administered in one or more portions, according to the available pharmaceutical dose unit.

The compounds of the invention can also be administered parenterally. In that case, the compounds are generally administered at the dosage regimen of 3 mg to 100 mg per day per Kg of body weight, more preferably of 5 mg to 50 mg per day per Kg of body weight. The daily dose can be also administered in one or more portions.

A composition containing a compound of the invention is suitably dispensed in unitary pharmaceutical dose form to simplify its administration and to achieve uniformity of the dispensed dosage. Accordingly, each solid dose unit for oral administration may contain a quantity of compound varying from 50 mg to 1000 mg, preferably from 200 to 500 mg.

In case of oral liquid forms, for a better compliance, the above dose units are dissolved in a volume of solvent multiple of 1.0 ml, such as 5.0 or 10.0 ml. The volumes may be then determined by using specific and currently available devices.

The parenteral dose unit of the compounds of the invention may contain a quantity of 100 mg to 1500 mg, preferably of 200 mg to 1000 mg. The parenteral unit may contain the compound already dissolved or as lyophilized powder, to be dissolved at the time of use in the prescribed amount of a suitable solvent.

The above dose units are produced with the conventional techniques already known to the skilled artisan and described in specialized publications, like “Remington: The Science and Practice of Pharmacy”, 20.th Edition, 2000.

According to another aspect, the present invention may relate to a use of an effective amount of at least one compound of formula (I) as defined above for the preparation of a pharmaceutical composition for the treatment of a disease associated with abnormal cell proliferation.

Because of the experimentally proved inhibitory activity on cell proliferation, the compounds of the invention are suitable for use in the preparation of a pharmaceutical composition for treating a related variety of diseases in a variety of conditions. In this regard, “treatment” or “treating” include both therapeutic and prophylactic treatments.

Accordingly, the compounds of the invention may be used to prepare a pharmaceutical composition for treating at very early stages of a disease, or even before early onset, or after significant progression, including metastasis. The terms “treatment” and “treating” are used to designate in particular a reduction of the burden in a patient, such as a reduction in cell proliferation rate, a destruction of diseased proliferative cells, a reduction of tumour mass or tumour size, a delaying of tumour progression, a reduction of number and size of metastases, as well as a complete tumour suppression.

Typical examples of diseases associated with abnormal cell proliferation include several types of cancer, for instance. The pharmaceutical compositions comprising compounds of the invention are particularly suited for treating several types of cancer, such as solid tumours or lymphoid tumours. Specific examples of the above pathological conditions include breast cancer, ovarian cancer, prostate cancer, bladder cancer, lung cancer, head and neck cancer, non-Hodgkin's lymphoma, melanoma, colon cancer, pancreas and liver cancers, and the like.

The invention is further illustrated for a better understanding in the following Examples, which shall not be considered limitative of the subject matter and of claims of the instant invention.

All starting product and reagents described in these examples are commercially available or, if not available, they have been manufactured by internal methods according to state of the art, but they are not a claimed part of this invention. These materials have been suitably stored according to suppliers/manufacturers' instructions supplied at the time of the delivery. Fresh aliquots were used for each modification.

### DESCRIPTION OF THE FIGURES

FIG. 1: Purification No. 1/a-Compound (I/A) crude lot A-2004/GR1-Chromatogram UV (214 nm, 260 nm, 280 nm) (Synthesis n. 1).

FIG. 2: Purification No. 2/a-Compound (I/A) crude lot A-2004/GR2-Chromatogram UV (214 nm, 260 nm, 280 nm) (Synthesis n. 2).

FIG. 3: Purification No. 3/a-Compound (I/A) crude lot B-2005/GR3-Chromatogram UV (214 nm, 260 nm, 280 nm) (Synthesis n. 3).

FIG. 4: Purification No. 4/a-Compound (I/A) crude lot C-2005/GR4-Chromatogram UV (214 nm, 260 nm, 280 nm) (Synthesis n. 4).

FIG. 5: Electrophoresis on polyacrylamide gel 10%-Purification No. 1/a-Compound (I/A) crude lot A-2004/GR1 and fractions A, B, C, D.

FIG. 6: Electrophoresis on polyacrylamide gel 15%-Purification No. 1/a-Compound (I/A) crude lot A-2004/GR1 and fractions A, B, C, D.

FIG. 7: Electrophoresis on polyacrylamide gel 15%-Compound (I/A) (fraction A) of crude lot B-2005/GR3 and rHLys.

FIG. 8: Electrophoresis on polyacrylamide gel 15%-Compound (I/A) crude (lots B-2005/GR3; C-2005/GR4) and purified (lots B-2005; C-2005) and rHLys.

FIG. 9: Purification No. 5-Compound (I/D) crude lot D-2005/GR5 Chromatogram UV (214 nm, 260 nm, 280 nm) (Synthesis n. 5).

FIG. 10: Electrophoresis on polyacrylamide gel 15%-Compound (I/D) crude (lots D-2005/GR5; E-2005/GR6; F-2005/GR7), purified (lots D-2005; E-2005; F-2005), fractions B (lots E-2005/GR6; F-2005/GR7) and rHLys.

FIG. 11: Purification No. 8-Compound (I/C) crude lot G-2005/GR8-Chromatogram UV (214 nm, 260 nm, 280 nm) (Synthesis n. 8).

FIG. 12: Electrophoresis on polyacrylamide gel 15%-Compound (I/C) purified lot G-2005 (fraction A), fractions B and C, and rHLys.

FIG. 13: Electrophoresis on polyacrylamide gel 15%-Compound (I/C) crude (lots H-2005/GR9; I-2005/GR10), purified (lots H-2005; I-2005), and rHLys.

FIG. 14: Purification No. 12/a-Compound (I/B) crude lot M-2005/GR12-Chromatogram UV (214 nm, 260 nm, 280 nm) (Synthesis n. 12).

FIG. 15: Electrophoresis on polyacrylamide gel 15%-Compound (I/B) purified lots M-2005/1 and M-2005/2 (fractions A) and fractions B.

FIG. 16: Calibration curve plotted with rHLys reference solution (Table n. 11/A).

FIG. 17: Calibration curve plotted with rHLys reference solution (Table n. 12/A).

FIG. 18: Calibration curve plotted with rHLys reference solution (Table n. 13/A).

FIG. 19: Calibration curve plotted with rHLys reference solution (Table n. 19/A).

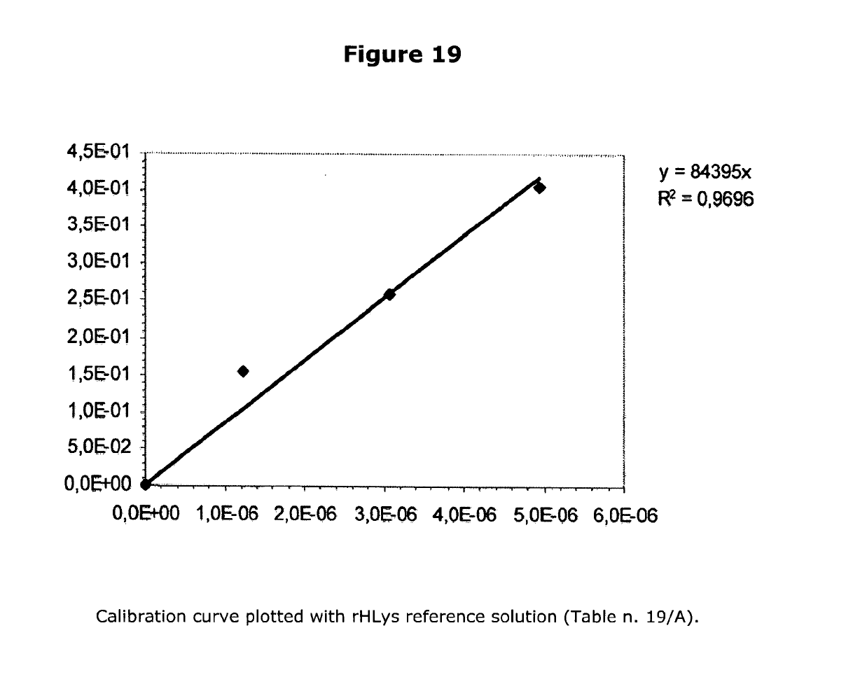

FIG. 20: Calibration curve plotted with rHLys reference solution (Table n. 26/A).

FIG. 21: Calibration curve plotted with rHLys reference solution (Table n. 30/A).

### 6. GENERAL ANALYTICAL METHODS FOR THE COMPOUNDS OF THE INVENTION

The analytical methods used for the analyses of the compounds of the invention and of their fractions, as reported in the following Examples, are described hereby:
(1) Ultraviolet light (UV) absorption chromatogram at 3 different wave lengths: 214 nm, 260 nm and 289 nm;
(2) Electrophoresis on polyacrylamide gel (10% and 15%);
(3) TNBS Test (TNBS=2,4,6-trinitrobenzenesulfonic acid) for the determination of free amino groups;
(4) Lowry Test for the determination of the Molecular Weight.

Despite the methods are known to a skilled artisan, for a better convenience they are briefly described here below:

### (1) Ultraviolet Light (UV) Absorption Chromatogram

The samples have been measured by determining the ultraviolet light (UV) absorption chromatograms at 3 different wave lengths: 214 nm, 260 nm and 280 nm.

### (2) Electrophoresis on Polyacrylamide Gel at 10% and 15%

The used technique is SDS-PAGE is carried out by using a 10% or 15% polyacrylamide gel. The gel is developed with 0.1% of Coomassie brilliant blue R-250 in methanol 45.0% and treated with 10.0% acetic acid until the desired level of coloration is obtained.

### (3) TNBS Test

The 0.03 M TNBS (2,4,6-trinitrobenzenesulfonic acid) solution is obtained withdrawing a 60 microlitres aliquot from a commercial solution (1 M) of TNBS, which is then introduced into a 2.0 ml volumetric flask, is brought to volume by using a pH 9.3 borate buffer solution.

Both the blank and the test solution of the sample to be tested are prepared according to the following method:
blank: 240 microlitres of the 0.03 M TNBS solution are introduced into a 10.0 ml volumetric flask and brought to volume with pH 9.3 borate buffer;
sample solution: a small aliquot (about 5.0 mg) of the compound under testing is accurately weighed and dissolved into a 10.0 ml volumetric flask with a small aliquot of pH 9.3 borate buffer; 250 microlitres of 0.03 M TNBS solution are added and the resulting mixture is brought to volume with pH 9.3 borate buffer. After 30 minutes the absorbance at 421 nm is determined and by using this method the percentage of free amino groups in the compound is calculated in relation to the blank.

### (4) Lowry Test

The method takes advantage on the formation of a coloured complex Cu⁺⁺-protein. The rameic ions in an alkaline solution of tartrate are able to coordinate the nitrogen atoms from the peptidic bonds. The subsequent addition of the phenolic reagent yields a blue-violet complex which absorbs between 550 and 750 nm proportionally to the total proteic concentration. In order to perform the test, at the time of use the following reagents are admixed:
Reagent A: sodium bicarbonate 2.0%+sodium tartrate 4.0%+CuSO₄ (copper sulfate) at the ratio 100:1:1;
Reagent B: Follin-Ciocalteau reagent diluted in water at the ratio 1:1.

In order to yield the reference solutions at known concentration, the following Table n. 1 is used.

TABLE n. 1
Procedure for the preparation of the reference solutions.

| | | Reference | Reference | Reference |
|---|---|---|---|---|
| Product | Blank | solution 1 | solution 2 | solution 3 |
| Sample | — | x ml | 2x ml | 3x ml |
| of starting | | | | |
| peptide | | | | |
| Bidistilled water | 0.5 ml | 0.5 ml-x ml | 0.5 ml-2x ml | 0.5 ml-3x ml |
| Reagent A | 2.0 ml | 2.0 ml | 2.0 ml | 2.0 ml |

After 10 minutes

| | | | | |
|---|---|---|---|---|
| Reagent B | 0.2 ml | 0.2 ml | 0.2 ml | 0.2 ml |

The sample is mixed with the prescribed quantities of water and of Reagent A. The mixture is left standing during 10 minutes at room temperature and then 0.2 ml of Reagent B is added; after stirring, the absorbance at 750 nm after 30 minutes from the last addition is determined. In order to yield a suitable calibration curve, at least three reference solutions at known content are prepared by using increasing quantities of the starting protein (rHLys) at known molecular weight (MW).

### 7. EXAMPLES

### Example 1

### Syntheses of Compound (I/A) via TmPEG 5 KDa

[TmPEG 5 KDa=Activated reagent (I)=methoxypolyethylene-glycol tresylate (mPEG=MW about 5 KDa)]

### Synthesis n. 1

1.0 g of rHLys were weighed and introduced into a 250.0 ml metered flask; 100 ml of sodium tetraborate pH 9.20 were added. Thereafter 2.01 g of commercial TmPEG 5 KD were added and the mixture was kept under stirring during 8 hours and at a temperature of 4° C. on ice bath. The solution was then brought to pH 4.5 with 100.0% acetic acid. The final solution was lyophilized to yield a quantity of 5.66 g of crude Compound (I/A), attributed lot number A-2004/GR1.

### Synthesis n. 2

5.0 g of rHLys were weighed and introduced into a 1 litre flask; 500 ml of sodium tetraborate pH 9.20 were added. A quantity of 10.0 g of commercial TmPEG 5 KDa were weighed and added. The reaction mixture was kept under stirring during 8 hours and at a temperature of 4° C. on ice water bath. Finally the solution was brought to pH 4.5 with 100% acetic acid. The solution was divided in to 10 flasks of 250.0 ml and then lyophilized to yield a quantity of 27.47 g of crude Compound (I/A), assigned lot A-2004/GR2.

### Synthesis n. 3

The synthesis has been repeated with further 5.01 g of rHLys by using the same quantities of sodium tetraborate pH 9.20 (500 ml) and commercial TmPEG 5 KDa (10.01 g). The reaction mixture was stirred during 8 hours and kept at a temperature of 4° C. on iced water bath. Finally the solution was brought to pH 4.5 by using 100.0% acetic acid, equally divided into 10 volumetric flasks of 250.0 ml and then lyophilized thus obtaining a total quantity of 28.85 g of crude Compound (I/A), assigned lot B-2005/GR3.

### Synthesis n. 4 (Excess of TmPEG 1.5:1

1.01 g of rHLys were weighed and introduced into a 250.0 ml volumetric flask; 100.0 ml of sodium tetraborate pH 9.20 were added. A quantity in excess (1.5:1-molar ratio 1:9) equivalent to 3.02 g of commercial TmPEG 5 KDa were weighed and added. The mixture was stirred during 8 hours and keep a temperature of about 4° C. on iced water bath. At the end the solution was brought to pH 4.5 by using 100.0% acetic acid. The solution was divided in two aliquots into 250.0 ml volumetric flasks. The solutions in the flasks were frozen by using the cold finger and lyophilized thus obtaining a total quantity of 6.44 g of crude Compound (I/A), assigned lot C-2005/GR4.

### Yields of the Syntheses of Crude Compound (I/A)

The yields of the 4 syntheses of crude Compound (I/A) have been summarized in Table n. 2.

TABLE n. 2
Yields of the syntheses of crude Compound (I/A).

| | | | Compound (I/A) | |
|---|---|---|---|---|
| | Product | Used | crude + other | Assigned lot |
| Syntheses | rHLys | TmPEG | fractions | n. |
| n. 1 | 1.00 g | 2.01 g | 5.66 g | A-2004/GR1 |
| n. 2 | 5.00 g | 10.00 g | 27.47 g | A-2004/GR2 |
| n. 3 | 5.01 g | 10.01 g | 28.85 g | B-2005/GR3 |
| n. 4 | 1.01 g | 3.02 g | 6.44 g | C-2005/GR4 |

### Example 2

Purification in Column of Crude Compound (I/A).

### Purification No. 1/a of Lot A-2004/GR1

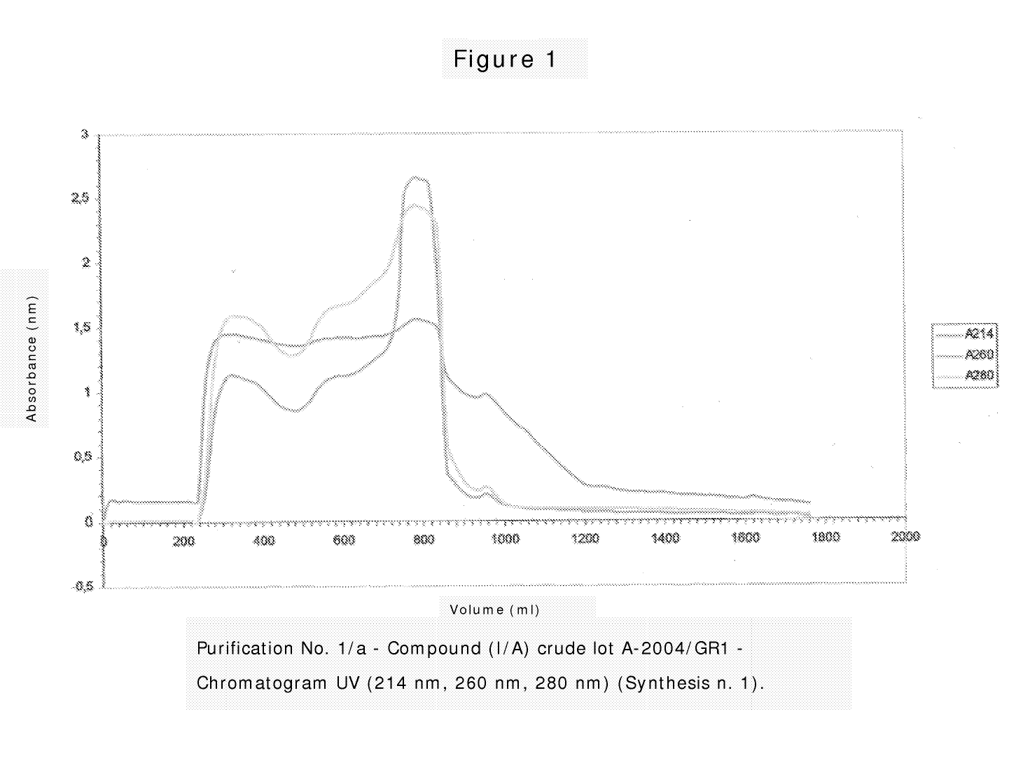

The used column (150.0×3.0 cm) was filled with the molecular screen resin SEPHADEX G-75 and acetic acid at 0.2% was used as eluent. 1.01 g of crude Compound (I/A) lot A-2005/GR1 were dissolved into 5.0 ml of eluent and loaded in column. The collected fractions, each of 20.0 ml, were analyzed at the UV light at three different wavelengths: 214 nm, 260 nm and 280 nm. The recorded chromatogram (FIG. 1) is enclosed as a reference also for the subsequent purifications of Compound (I/A). The collected fractions were divided into four aliquots: A, B, C and D, which were lyophilized separately and thereafter analyzed by means of electrophoresis on polyacrylamide gel. From the lyophilization of fraction A, 0.119 g of Compound (I/A), assigned lot number A-2004/1a, were obtained. A residue of 0.180 g was obtained from the lyophilization of fraction B, and 0.042 g from fraction C. The residue of aliquot D resulted 0.513 g.

### Purification No. 1/b of Lot A-2004/GR1

The remaining quantity of 4.65 g of crude Compound (I/A) lot A-2004/GR1 have been purified by using the same method and conditions described at the previous section. The relevant fractions have been divided, collected and lyophilized with the same method.

1.036 g of purified Compound (I/A), identified as lot A-2004/1b, were obtained from fraction A. Fraction B contained 0.967 g of partially reacted starting product. The aliquots C (not reacted starting product) and D (salts) were discarded. Lots A-2004/1a and A-2004/1b of purified Compound (I/A) have been admixed to yield one batch only of 1.155 g, with assigned lot number A-2004/1.

### Purification No. 2/a of Lot A-2004/GR2

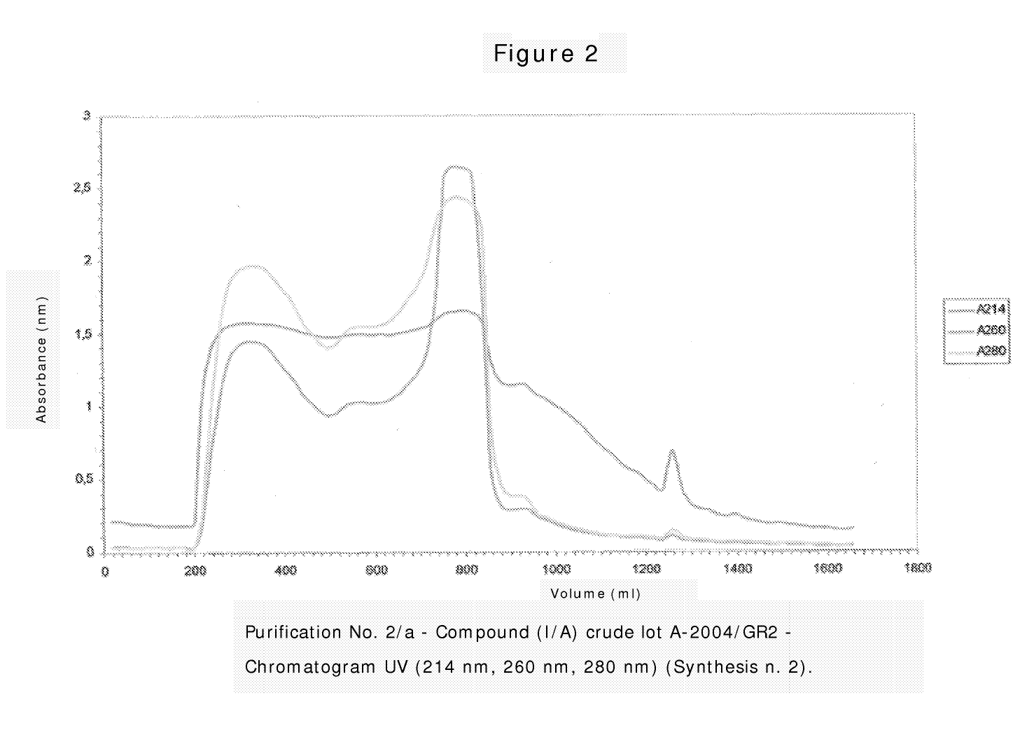

5.1 g of crude Compound (I/A) lot A-2004/GR2 was loaded into the column dissolved into 24.0 ml of acetic acid 0.2%. The relevant chromatogram has been recorded (FIG. 2). 1.136 g of purified Compound (I/A), identified as lot A-2004/2a, was obtained from the fraction A, while 0.940 g were obtained from the remaining fraction B, being partially reacted starting product.

### Purifications No. 2/b, 2/c, 2/d and 2/e of Lot A-2004/GR2

The remaining four aliquots of crude Compound (I/A) lot A-2004/GR2 were separately loaded into columns after dissolving each aliquot into 24.0 ml of acetic acid 0.2%, purified with the same method herein above described and identified with the lot number reported in the subsequent Table n. 3. The five fractions A, resulting from the individual purifications (total 5.910 g), were mixed together (lot A-2004/2) and poured into a 500 ml flask with 125 ml of bidistilled water. The solution was lyophilized to yield 5.450 g of purified Compound (I/A), assigned lot number A-2004.

### Purification No. 3/a of Lot B-2005/GR3

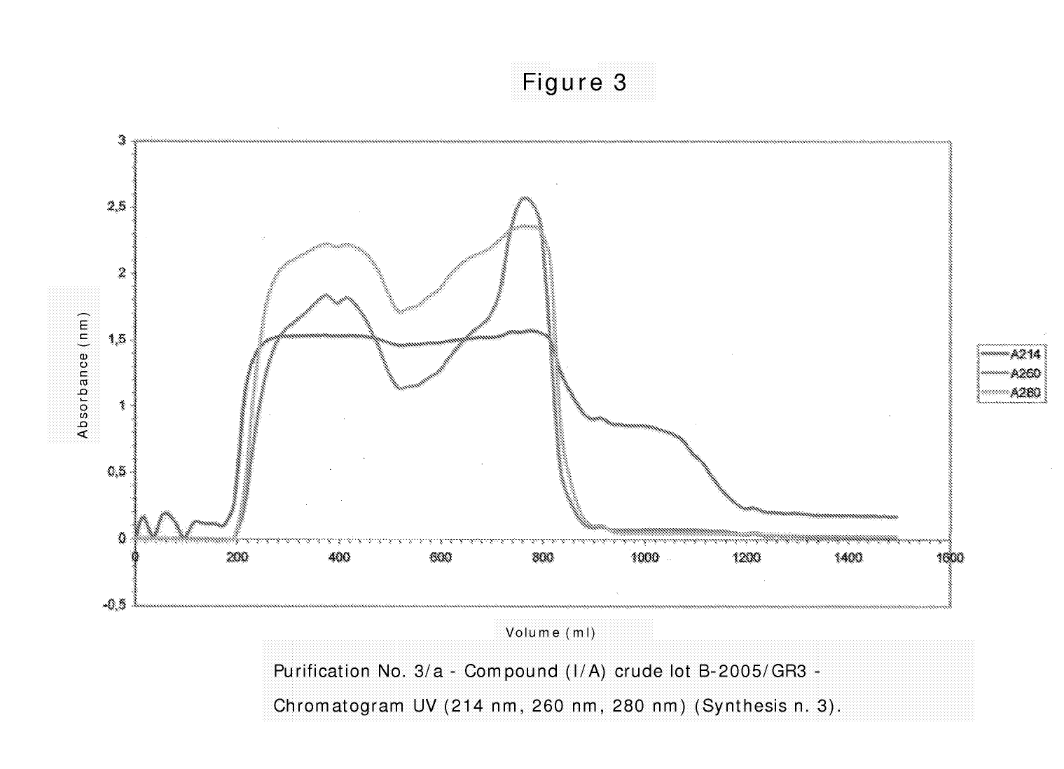

By using the same conditions described for the previous purifications, 5.002 g of crude Compound (I/A) lot B-2005/GR3 was loaded into a column dissolved into 24.0 ml of acetic acid 0.2%. The relevant UV chromatogram (FIG. 3) has been recorded and is enclosed for reference. 1.274 g of purified Compound (I/A) with the assigned lot number B-2005/3a have been obtained.

### Purifications No. 3/b, 3/c and 3/d of Lot B-2005/GR3

The remaining three aliquots of crude Compound (I/A) lot B-2005/GR3 were separately loaded into columns after dissolution into 24.0 ml of acetic acid 0.2%, purified with the same method herein above described and identified with the lot number reported in the subsequent Table n. 3.

### Purification No. 4/a of Lot C-2005/GR4

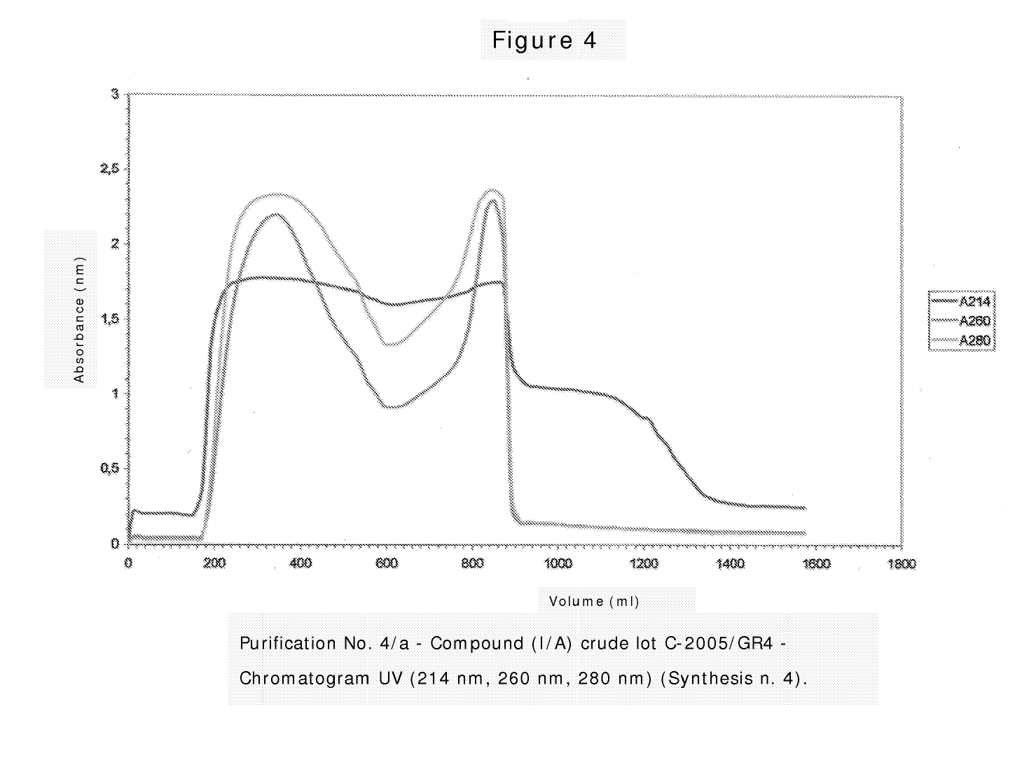

By using the same conditions described for the previous purifications, 5.0 g of crude Compound (I/A) lot C-2005/GR4 was dissolved into 24.0 ml of acetic acid 0.2% and loaded into a column. Fractions have been divided by using the herein above described technique. The relevant UV chromatogram (FIG. 4) has been recorded. A total of 1.46 g of purified Compound (I/A) lot number C-2005 has been obtained.

### Purification Yields of Crude Compound (I/A)

The yields of the purifications of crude Compound (I/A) have been summarized in Table n. 2.

TABLE n. 3
Purifications of crude Compound (I/A).

| | Compound (I/A) | | Purified | |
|---|---|---|---|---|
| Purification | crude + other | Lot n. | Compound (I/A) | Assigned lot |
| No. | fractions | Crude | (fraction A) | n. |
| Purification 1/a | 1.010 g | A-2004/GR1 | 0.119 g | A-2004/1a |
| Purification 1/b | 4.650 g | A-2004/GR1 | 1.036 g | A-2004/1b |
| Purifications 1 | Combined | | 1.155 g | A-2004/1 |
| | Purifications 1/a and 1/b | | | |
| Purified | Result of final lyophilization | | 1.155 g | A-2004/1 |
| Compound (I/A) | of Purifications 1 | | | |
| Purification 2/a | 5.100 g | A-2004/GR2 | 1.136 g | A-2004/2a |
| Purification 2/b | 5.005 g | A-2004/GR2 | 1.225 g | A-2004/2b |
| Purification 2/c | 5.007 g | A-2004/GR2 | 1.081 g | A-2004/2c |
| Purification 2/d | 5.005 g | A-2004/GR2 | 1.263 g | A-2004/2d |
| Purification 2/e | 5.005 g | A-2004/GR2 | 1.205 g | A-2004/2e |
| Purifications 2 | Purifications 2/a, 2/b, 2/c, 2/d | | 5.910 g | A-2004/2 |
| | and 2/e combined | | | |
| Purified | Result of final lyophilization | | 5.450 g | A-2004 |
| Compound (I/A) | of Purifications 2 | | | |
| Purification 3/a | 5.002 g | B-2005/GR3 | 1.274 g | B-2005/3a |
| Purification 3/b | 5.000 g | B-2005/GR3 | 1.384 g | B-2005/3b |
| Purification 3/c | 5.003 g | B-2005/GR3 | 1.385 g | B-2005/3c |
| Purification 3/d | 5.002 g | B-2005/GR3 | 1.322 g | B-2005/3d |
| Purifications 3 | Purifications 3/a, 3/b, 3/c and | | 5.365 g | B-2005/3 |
| | 3/d combined | | | |
| Purified | Result of final lyophilization | | 5.047 g | B-2005 |
| Compound (I/A) | of Purifications 3 | | | |
| Purification 4/a | 5.000 g | C-2005/GR4 | 1.460 g | C-2005 |

| | | | |
|---|---|---|---|
| Purified | None final lyophilisation | 1.460 g | C-2005 |
| Compound (I/A) | | | |

### Example 3

### Compound (I/A)

### Examples of Analyses of Crude and Purified Fractions

Lots of Compound (I/A) (fraction A) and other obtained fractions have been analyzed according to the following already described methods:
Electrophoresis on polyacrylamide gel at 10% and 15%;
TNBS Test to determine the free amino groups;
Lowry Test to determine the molecular weight.

### 1-A) Electrophoresis on Polyacrylamide Gel at 10% and 15%

Compound (I/A) lot A-2004/GR1 and Purified A-2004/1

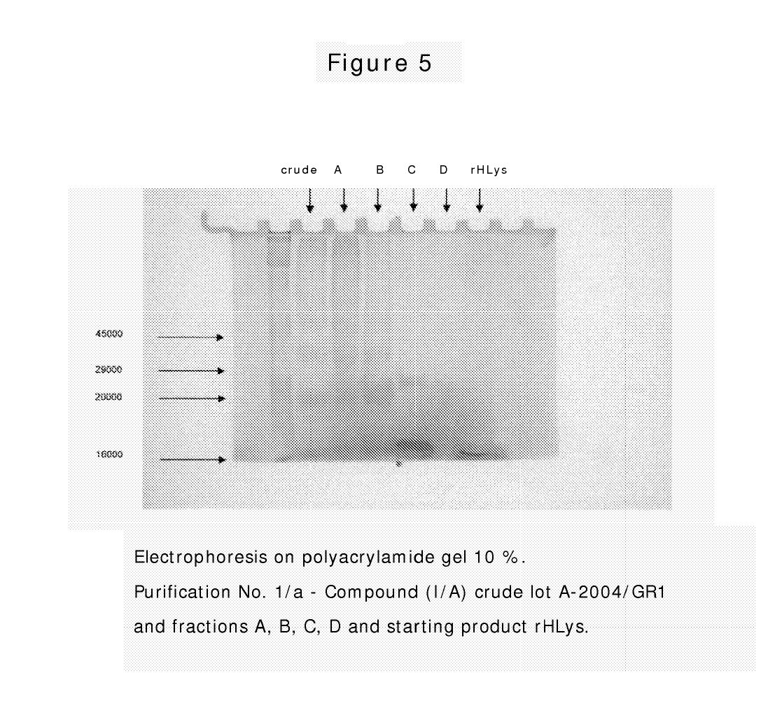

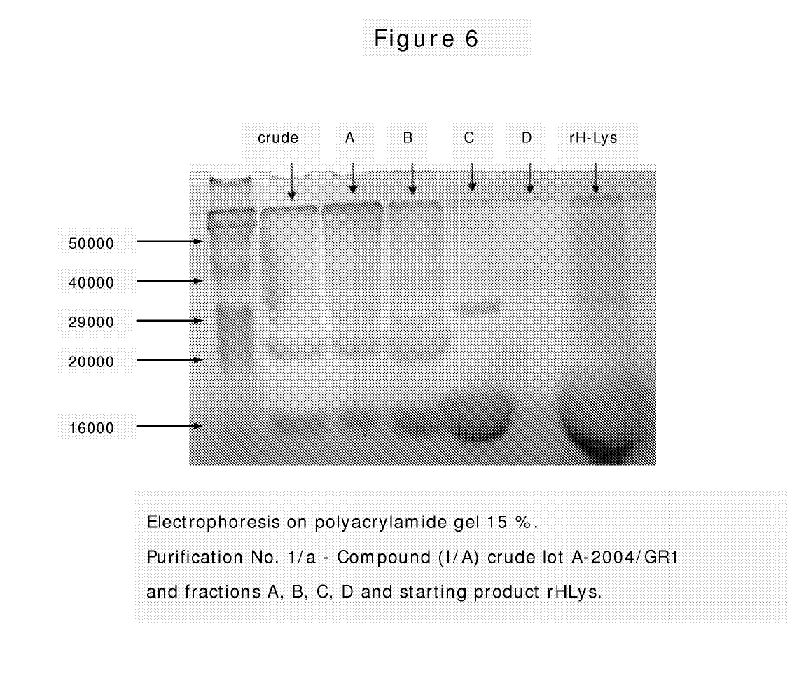

The support for electrophoresis on polyacrylamide gel has been prepared according to the described general method (SDS-PAGE technique). 5 mg of crude Compound (I/A) lot A-2004/GR1, 5 mg of each single fraction separated in column (identified as fractions A, B, C and D) and 5 mg of rHLys have been weighed and then each dissolved separately in aliquots of 50 microlitres of bidistilled water. From the electrophoresis on polyacrylamide gel at 10% (FIG. 5) and at 15% (FIG. 6) the fraction A, consisting of purified Compound (I/A) lot A-2004/1, resulted to be the most pegylated, B was partially pegylated, while fraction C presented the higher presence of the starting product rHLys; fraction D was substantially containing salts.

### 1-B) Electrophoresis on Polyacrylamide Gel at 15%

Compound (I/A) Lot B-2005/GR3

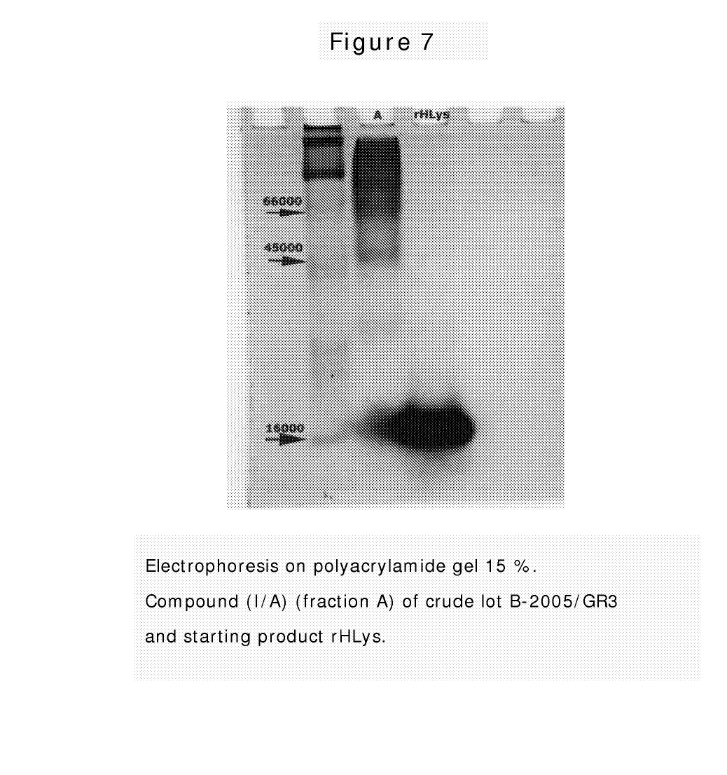

By using the above described technique, 5 mg of Compound (I/A) (fraction A) obtained from the crude lot B-2005/GR3 and 5 mg of the starting peptide rHLys have been weighed and they have been separately dissolved in 50 microlitres of bidistilled water, then performing the electrophoresis on polyacrylamide gel at 15% (FIG. 7).

### 1-C) Electrophoresis on Polyacrylamide Gel at 15%

Compound (I/A) Crude (Lots B-2005/GR3; C-2005/GR4) and Purified (Lots B-2005; C-2005) and rHLys.
By using the already described technique, 5 mg of each sample under testing has been weighed, as indicated in the subsequent Table n. 4. Each sample has been then dissolved in 500 microlitres of bidistilled water and then applied on the plate for electrophoresis.

TABLE n. 4
Tested samples and corresponding identification codes.

| Code | Sample | Source |
|---|---|---|
| R | References of known molecular weight | — |
| | (16-20-30-46 KDa) | |
| 1GR | B-2005/GR3 | Synthesis n. 3 |
| 1A | B-2005/3 (fraction A) | Purifications No. 3 |
| 1B | fraction B (B-2005/GR3) | Purifications No. 3 |
| 2GR | C-2005/GR4 | Synthesis n. 4 |
| 2A | C-2005 (C-2005/4a - fraction A) | Purification No. 4a |
| 2B | fraction B (C-2005/GR4) | Purification No. 4a |
| rHLys | Recombinant Human Lysozyme | — |

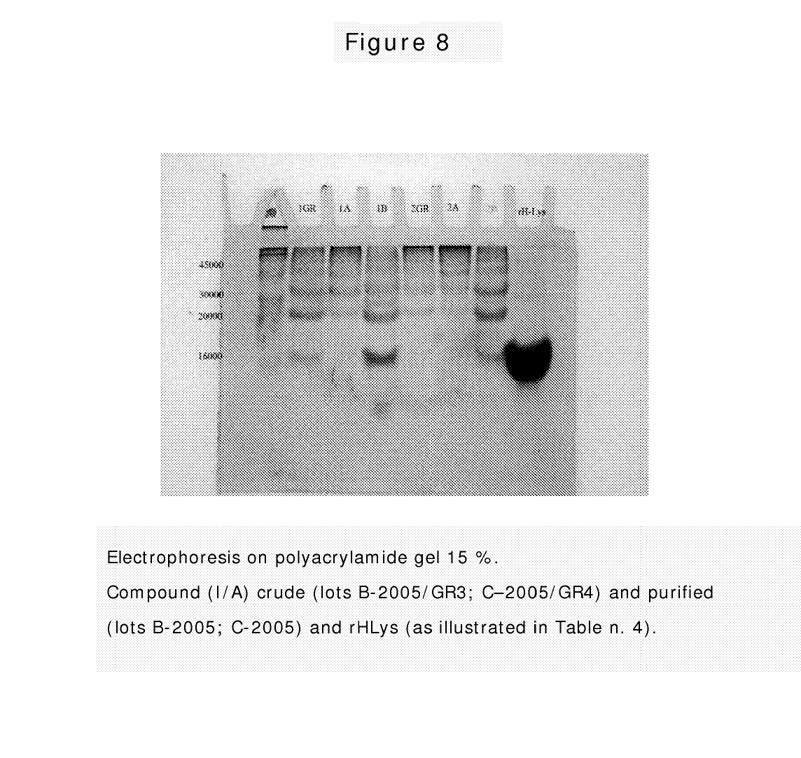

From the electrophoresis (FIG. 8) it is resulting that lots B-2005 and C-2005, consisting of fractions A, are the most rich of highly pegylated Compound (I/A), while the corresponding fractions B are consisting of partially pegylated products and of starting product.

### 2-A) TNBS Test—Purified Compound (I/A) Lot A-2004/2

The quantities indicated in Table n. 5 of the reference product rHLys and of the purified Compound (I/A) lot A-2004/2 have been weighed and poured into separate 10.0 ml volumetric flasks. The samples have been dissolved in a minimum aliquot of borate buffer pH 9.3, 250 microlitres of 0.03 M TNBS have been added and the mixture has been brought to volume with borate buffer. After 30 minutes the absorbance at 421 nm has been determined and the obtained figures have been reported in Table n. 5.

TABLE n. 5

| Product | Weighed quantity | A 421 nm |
|---|---|---|
| Staring peptide (rHLys) | 5.1 mg | 0.202 |
| Compound (I/A) purified | 5.1 mg | 0.108 |
| (Fraction A) lot A-2004/2 | | |

Table n. 6 summarizes the percentage of free amino groups in the purified Compound (I/A) (fraction A) lot A-2004/2.

TABLE n. 6
Percentage of free amino groups (—NH₂).

| Product | % of free amino groups |
|---|---|
| Compound (I/A) purified | 13.10% |
| (Fraction A) lot A-2004/2 | |

### 2-B) TNBS Test—Purified Compound (I/A) Lot B-2005

The quantities indicated in Table n. 7 of the reference product rHLys and of the purified Compound (I/A) lot B-2005 have been weighed and poured into separate 10.0 ml volumetric flasks. It has been proceeded as indicated before.

TABLE n. 7
Weighed quantities and corresponding values of absorbance at 421 nm.

| Product | Weighed quantity | A 421 nm |
|---|---|---|
| Starting peptide (rHLys) | 5.1 mg | 0.289 |
| Compound (I/A) purified | 5.1 mg | 0.101 |
| (fraction A) lot B-2005 | | |

The percentage of free amino groups has been summarized in Table n. 8.

TABLE n. 8
Percentage of free amino groups (—NH₂).

| Product | % free amino groups |
|---|---|
| Compound (I/A) purified | 8.73% |
| (fraction A) lot B-2005 | |

### 2-C) TNBS Test—Purified Compound (I/A) Lot C-2005

The quantities indicated in Table n. 9 of the reference product rHLys and of the purified Compound (I/A) lot C-2005 have been weighed and poured into separate 10.0 ml volumetric flasks. It has been proceeded as indicated before.

TABLE n. 9
Weighed quantities and corresponding
figures of absorbance at 421 nm.

| Product | Weighed quantity | A 421 nm |
|---|---|---|
| Starting peptide (rHLys) | 5.1 mg | 0.378 |
| Compound (I/A) purified | 5.1 mg | 0.103 |
| (fraction A) lot C-2005 | | |

The percentage of free amino groups were summarized in Table n. 10.

TABLE n. 10
Percentage of free amino groups (—NH₂).

| Product | % free amino groups |
|---|---|
| Compound (I/A) purified | 6.81% |
| (fraction A) lot C-2005 | |

### 3-A) Lowry Test—Purified Compound (I/A) Lot A-2004/2

Lowry Test has been performed on purified Compound (I/A) lot A-2004/2. Two solutions were prepared: one reference, being in this specific case 5.1 mg of reference peptide rHLys dissolved in 10.0 ml of bidistilled water, to plot the calibration curve, and the second of purified Compound (I/A) lot A-2004/2 (fraction A) at the same concentration of 5.1 mg in 10.0 ml. These quantities were poured separately in 10.0 ml volumetric flask and brought to volume with bidistilled water.

From these solutions were then withdrawn different quantities, as indicated in Table n. 11, which have been poured into 5.0 ml volumetric flasks.

TABLE n. 11
Table inherent the Lowry Test.

| | | | | | | Purified |
|---|---|---|---|---|---|---|
| | | | | | Crude | Compound |
| Product | Blank | Reference 1 | Reference 2 | Reference 3 | Compound | (fract. A) |
| Sample | — | 0.2 ml | 0.5 ml | 0.8 ml | 0.8 ml | 0.8 ml |
| Bidistilled | 0.926 ml | 0.726 ml | 0.426 ml | 0.126 ml | 0.126 ml | 0.126 ml |
| water | | | | | | |
| Reagent A | 3.704 ml | 3.704 ml | 3.704 ml | 3.704 ml | 3.704 ml | 3.704 ml |

After 10 minutes

| | | | | | | |
|---|---|---|---|---|---|---|
| Reagent B | 0.37 ml | 0.37 ml | 0.37 ml | 0.37 ml | 0.37 ml | 0.37 ml |

After reading the absorbance at 750 nm of the reference solutions (reference peptide rHLys), a calibration curve was plotted (the concentration in the abscissa and the absorbance in the ordinate). From the above the value of ε has been then calculated, in order to determine the molecular weight of the purified Compound (I/A) lot A-2004/2 (fraction A).

TABLE n. 11/A

| | C | A 750 nm |
|---|---|---|
| Reference 0 | 0 | 0 |
| Reference 1 | 1.23E−06 | 0.272 |
| Reference 2 | 3.08E−06 | 0.451 |
| Reference 3 | 4.94E−06 | 0.641 |

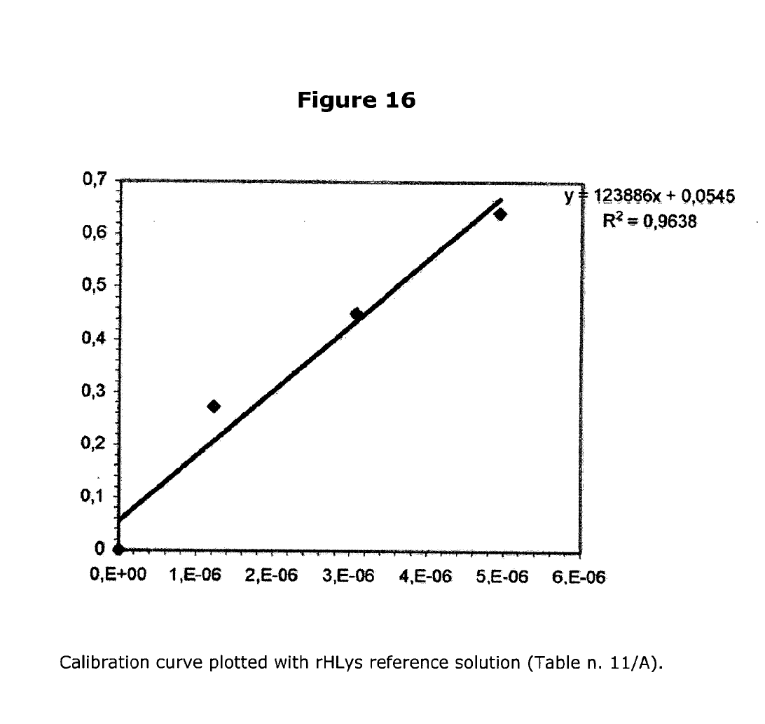

Calibration curve plotted with rHLys reference solutions is shown in FIG. 16.

Once the calibration curve was plotted with the figures of the absorbance of the concentrations of the reference solutions, the value of ε was calculated, in this case resulting 123886, by applying the equation: [MW=g/l*ε/A] and therefore the molecular weight (MW) of each single fraction was calculated. Therefore the molecular weight for the purified Compound (I/A) lot A-2004/2 (fraction A) resulted of about 46,000 Dalton.

The results were summarized in the following Table n. 12.

TABLE n. 12
Quantity of purified Compound (I/A) (fraction A) lot A-2004/2 in
solution, relevant absorbance and molecular weight.

| | Weight (g) | | |
|---|---|---|---|
| Product | in 5.0 ml | A 750 nm | MW (g/mole) |
| Compound (I/A) purified | 8.16E−02 | 0.293 | 34502 |
| (fraction A) lot A-2004/2 | | | |
| Calculated | | | ~ 46000 |

ε=123886 A=ε*C C=g/l*MW.

To determine the MW the following equation has been applied: MW=g/l*ε/A

### 3-B) Lowry Test—Purified Compound (I/A) Lot B-2005

A second example of Lowry Test has been carried out on purified Compound (I/A) lot B-2005. The reference solution to plot the calibration curve have been prepared according to the already described procedure. Two solutions were prepared: one reference, consisting in this specific case of 5.1 mg of reference peptide (rHLys) dissolved in 10.0 ml of bidistilled water (to plot the calibration curve) and the second of the purified Compound (I/A) lot B-2005, at the same concentration of 5.1 mg in 10.0 ml of bidistilled water. These quantities were poured separately in 5.0 ml volumetric flasks and brought to volume with bidistilled water. From these solutions were then withdrawn different quantities, as indicated the previous example. After reading the values of the absorbance at 750 nm of the reference solutions and of the purified Compound (I/A) lot B-2005, the calibration curve has been plotted (the concentration in the abscissa and the absorbance in the ordinate).

TABLE n. 12/A

| | C | A 750 nm |
|---|---|---|
| Reference 0 | 0 | 0 |
| Reference 1 | 1.23E−06 | 0.272 |
| Reference 2 | 3.08E−06 | 0.451 |
| Reference 3 | 4.94E−06 | 0.641 |

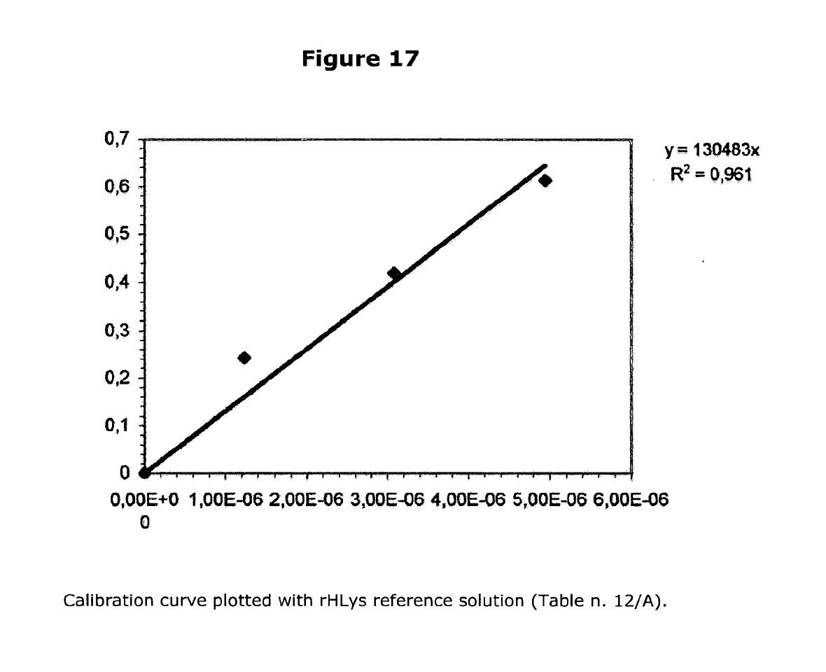

Calibration curve inherent to rHLys reference solutions is shown in FIG. 17. From the determined results a ε value of 130483 was calculated by applying the equation: [MW=g/l*ε/A].

Therefore for the purified Compound (I/A) lot B-2005 a MW of about 46,000 Dalton has been calculated.

The results were summarized in the following Table n. 13.

TABLE n. 13
Quantity of purified Compound (I/A) (fraction A) lot B-2005 in
solution, relevant absorbance and molecular weight.

| | Weight (g) | | |
|---|---|---|---|
| Product | in 5.0 ml | A 750 nm | MW (g/mole) |
| Compound (I/A) purified | 8.16E−02 | 0.24 | 44364 |
| (fraction A) lot B-2005 | | | |

ε=130483 A=ε*C C=g/l*MW.

To determine the MW the equation has been applied:

MW=g/l*ε/A

### 3-C) Lowry Test—Purified Compound (I/A) Lot C-2005

Lowry Test has been carried out on the purified Compound (I/A) lot C-2005 by using the same techniques already described in the previous examples. After reading the values of the absorbance at 750 nm of the reference solutions and of the purified Compound (I/A) lot C-2005, the calibration curve has been plotted (the concentration in the abscissa and the absorbance in the ordinate).

TABLE n. 13/A

| | C | A 750 nm |
|---|---|---|
| Reference 0 | 0 | 0 |
| Reference 1 | 1.23E−06 | 0.262 |
| Reference 2 | 3.08E−06 | 0.477 |
| Reference 3 | 4.94E−06 | 0.629 |

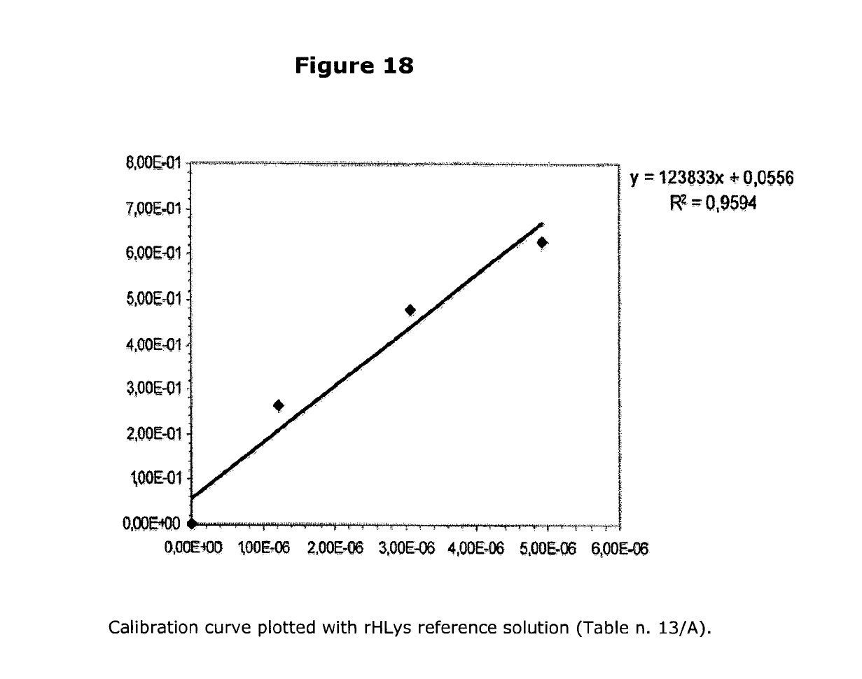

Calibration curve inherent rHLys reference solutions is shown in FIG. 18.

After plotting the calibration curve, the value of ε was calculated, being in this case 123833, and by applying the equation: MW=g/l*ε/A the experimental value of the presumed molecular weight has been determined.

The results were summarized in Table n. 14.

TABLE n. 14
Quantity of purified Compound (I/A) (fraction A) lot C-2005 in
solution, relevant absorbance and molecular weight.

| | Weight (g) | | |
|---|---|---|---|
| Product | in 5.0 ml | A 750 nm | MW (g/mole) |
| Compound (I/A) purified | 4.08E−04 | 0.156 | 64774 |
| (fraction A) lot C-2005 | | | |

ε=123883 A=ε*C C=g/l*MW

To determine the MW the equation has been applied:

MW=g/l*ε/A

### Conclusions of the Preceding Analytical Results

The tested lots of purified Compound (I/A) (fraction A) showed at the electrophoresis, at the TNBS Test and at the Lowry Test experimental results which were expected for the saturated conjugate, that is hexa-methoxypegylated-5 KDa, of the starting peptide rHLys. On the contrary fraction B was consisting of conjugates which partially reacted and that, presenting different molecular weights depending from the pegylation degree, may be selectively separated with techniques already known to a skilled artisan.

### Example 4

Synthesis of Activated Reagent (V) mPEG-OSu 5 KDa (mPEG-SC 5 KDa).

15.01 g of mPEG 5 KDa, namely methoxy-polyethylenglycol with MW=5 KDa (5,000 Daltons) (brand Fluka) were poured into 1 litre volumetric flask, dissolved in 60.0 ml of acetonitrile and placed into a rotavapor at 30° C. to complete the solvent evaporation. This operation was repeated again by using a lower volume (50.0 ml) of acetonitrile. The residue was dried by means of a vacuum pump during 1 hour. The obtained solid residue was dissolved again in 50.0 ml of acetonitrile and precipitated with 200.0 ml of ethyl ether under stirring and on ice bath. The solid was separated by filtration on a Gooch (porosity 3) and the precipitate was washed for at least 3 fold with ethyl ether. The Gooch with mPEG 5 KDa was transferred into a desiccator for 30-60 minutes. 100.0 ml of ethanol were warmed to 40° C. and added slowly to 1 litre flask in order to dissolve completely mPEG 5 KDa. The collected limpid solution was placed in a freezer during about 2 hours. After 2 hours mPEG 5 KDa was precipitated, so that it was filtered on Gooch (porosity 3) and washed with ethyl ether at least 3 fold. The Gooch was placed over night in a desiccator under vacuum. mPEG 5 KDa was then recovered from the desiccator, weighed (14.20 g), poured into a 250.0 ml volumetric flask and then dissolved with 50.0 ml of dichloromethane and evaporated in a rotavapor at 30° C. The solid was then finally dried during 30 minutes with a vacuum pump. Aliquots of 20.0 ml of acetonitrile, 10.0 ml of pyridine and 10.0 ml of acetonitrile were added to mPEG 5 KDa. Disuccimidyl-carbonate (2.18 g) was then added to activate mPEG 5 KDa, in the molar ratio 3:1, in relation to the moles of mPEG 5 KDa. The mixture was then stirred over night at room temperature.

The next day the activated reagent mPEG-OSu 5 KDa (namely known as mPEG-SC 5 KDa) was precipitated with ethyl ether and was dried in about 10-15 minutes. mPEG-OSu 5 KDa was then removed from the desiccator and poured into 1 litre volumetric flask. Warm ethanol (40° C.) was slowly added to yield a clear solution. The solution was stored during about 2 hours in refrigerator until a white precipitate of mPEG-OSu 5 KDa (mPEG-SC 5 KDa) appeared. The product was then removed from the refrigerator, separated by filtration, washed with ethyl ether and poured into a desiccator for at least one day. 14.15 g of activated reagent mPEG-OSu 5 KDa (mPEG-SC 5 KDa) were obtained.

### Example 5

Syntheses of Compound (I/D) via mPEG-OSu 5 KDa (mPEG-SC 5 KDa) [mPEG-OSu 5 KDa (namely mPEG-SC 5 KDa)=Activated reagent (V)=methoxypolyethylene glycol succinimidyl carbonate (mPEG=MW. about 5 KDa)]

Synthesis n. 5 (Excess 1.5:1 of mPEG-OSu)

1.01 g of starting peptide rHLys were weighed, poured into a 250.0 ml volumetric flask and added to 100.0 ml of pH 9.2 sodium tetraborate. 3.02 g of mPEG-OSu 5 Kda (obtained from Example 4) were weighed and poured in excess (1.5:1/molecular ratio 1:9) into the flask. The mixture was kept under constant stirring during 8 hours at 4° C. on ice bath. Finally the solution was brought to pH 4.5 with 100.0% acetic acid. The solution was equally divided in two 250 ml volumetric flasks. The solutions in the flasks were cooled by means of the cold finger and thus lyophilized, obtaining 7.01 g of crude Compound (I/D), assigned lot number D-2005/GR5.

### Synthesis n. 6 (Standard Conditions)

1.01 g of starting peptide (rHLys) were weighed, poured into a 250.0 ml volumetric flask and added to 100.0 ml of p 9.2 sodium tetraborate. 2.01 g of mPEG-OSu 5 KDa (obtained from Example 4) were weighed and poured into the flask. The mixture was kept under constant stirring during 8 hours at 4° C. on ice bath. Finally the solution was brought to pH 4.5 with 100.0% acetic acid. The solution was equally divided in two 250 ml volumetric flasks. The solutions in the flasks were cooled by means of the cold finger and lyophilized, thus obtaining 6.37 g of crude Compound (I/D), identified as lot E-2005/GR6.

Synthesis n. 7 (Excess 2:1 of mPEG-OSu 5 KDa)

1.01 g of starting peptide rHLys were weighed, poured into a 250.0 ml volumetric flask and added to 100.0 ml of ph 9.20 sodium tetraborate. 6.01 g of mPEG-OSu 5 KDa (obtained from Example 4) were weighed and poured in excess (2:1/molecular ratio 1:18) into the flask. The mixture was kept under constant stirring during 8 hours at 4° C. on ice bath. Finally the solution was brought to pH 4.5 with 100.0% acetic acid. The solution was equally divided in two 250 ml volumetric flasks. The solutions in the flasks were cooled by means of the cold finger and lyophilized, thus obtaining 10.47 g of crude Compound (I/D) with assigned lot number F-2005/GR7.

### Yields of the Syntheses of Crude Compound (I/D)

The yields of the 3 syntheses of crude Compound (I/D) have been summarized in Table n. 15.

TABLE n. 15
Yields of the syntheses of crude Compound (I/D).

| | | Used | Compound (I/D) | |
|---|---|---|---|---|
| | | mPEG-OSu | crude + other | |
| Syntheses | rHLys | 5 KDa | fractions | Lot n. |
| Synthesis n. 5 | 1.01 g | 3.02 g | 7.01 g | D-2005/GR5 |
| (excess 1.5:1/ | | | | |
| m.r. 1:9) (*) | | | | |
| Synthesis n. 6 | 1.01 g | 2.01 g | 6.37 g | E-2005/GR6 |
| (standard | | | | |
| conditions) | | | | |
| Synthesis n. 7 | 1.01 g | 6.01 g | 10.47 g | F-2005/GR7 |
| (excess 2:1/ | | | | |
| m.r. 1:18) (*) | | | | |

[(*) m.r. = molar ratio]

### Example 6

Purification in column of crude Compound (I/D).

### Purification No. 5 of Lot D-2005/GR5

The same conditions and techniques already described for the purifications of Compound (I/A) have been adopted.

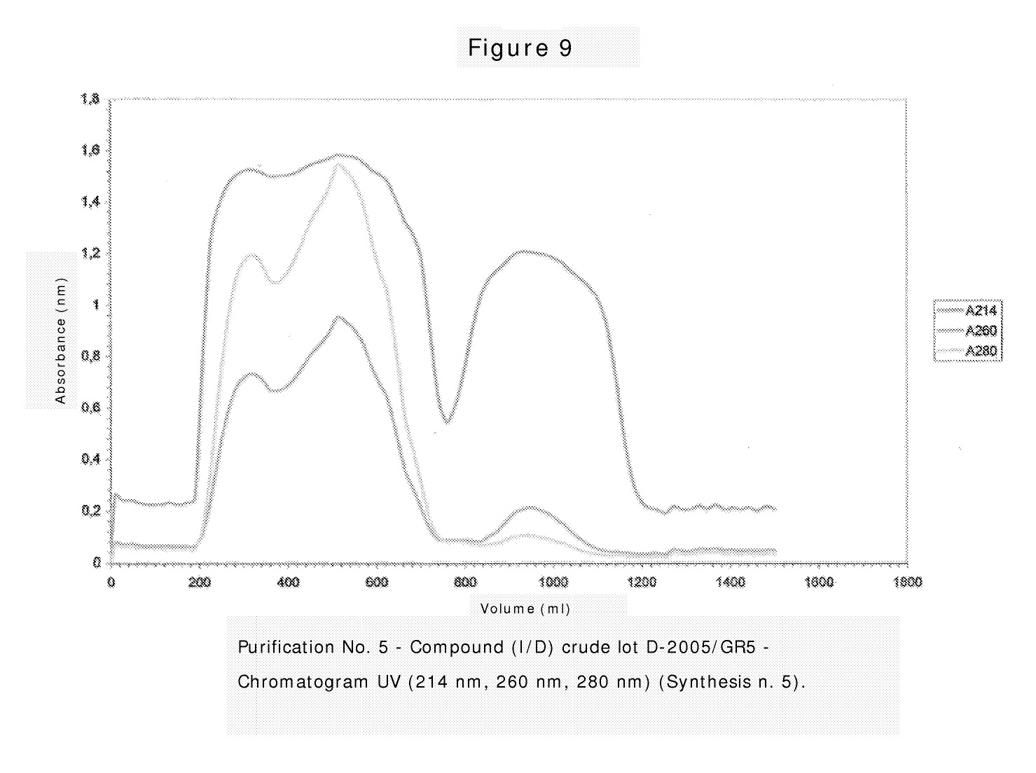

2.0 g of crude Compound (I/D) lot D-2005/GR5 were dissolved with 12.0 ml of acetic acid 0.2% and loaded into a column. The collected fractions, each of 20.0 ml, were analyzed at the UV light at three different wavelengths: 214 nm, 260 nm and 280 nm. The fractions of the Compound (I/D) and of starting peptide were grouped according to the results of the recorded chromatogram (FIG. 9), which is enclosed for reference. The collected fraction A was divided for the lyophilization into 6 aliquots, each of about 100.0 ml. Each aliquot was poured into a 250.0 ml volumetric flask, cooled and lyophilized. 1.0 g of purified Compound (I/D) Fraction A, with assigned lot number D-2005, has been obtained.

### Purification No. 6 of Lot E-2005/GR6

5.0 g of crude Compound (I/D) lot E-2005/GR6 were dissolved with 12.0 ml of acetic acid 0.2% and loaded into a column. The collected fractions, each of 20.0 ml, were analyzed at the UV light at three different wavelengths: 214 nm, 260 nm and 280 nm. The fractions of Compound (I/D) and of starting peptide rHLys were grouped according to the results of the recorded chromatogram. The collected fraction A was divided for the lyophilization into 6 aliquots, each of about 100.0 ml. Each aliquot was poured into a 250.0 ml volumetric flask, cooled and lyophilized. 1.44 g of purified Compound (I/D) Fraction A, lot E-2005, were obtained.

### Purification No. 7 of Lot F-2005/GR7

5.01 g of crude Compound (I/D) lot f-2005/GR7 were dissolved with 24.0 ml of the acetic acid 0.2% and loaded into a column. The collected fractions, each of 20.0 ml, were analyzed at the UV light at three different wavelengths: 214 nm, 260 nm and 280 nm. The collected fractions A of Compound (I/D) and of the starting peptide rHLys were grouped according to the results of the recorded chromatogram. The collected fraction A was divided for the lyophilization into 6 aliquots, each of about 100.0 ml. Each aliquot was poured into a 250.0 ml volumetric flask, cooled with the cold finger and lyophilized. 2.25 g of purified Compound (I/D) Fraction A, lot F-2005, were obtained.

### Purification Yields of Crude Compound (I/D)

The purifications yield of crude Compound (I/D) have been summarized in Table n. 16.

TABLE n. 16
Purification yields of crude Compound (I/D).

| | | | Compound | |
|---|---|---|---|---|
| | Compound | | (I/D) | |
| | (I/D) | Lot n. | purified (*) | Lot n. |
| Purifications | crude | (crude) | (fraction A) | (purified) |
| Purification No. 5 | 2.00 g | D-2005/GR5 | 1.00 g | D-2005 |
| Purification No. 6 | 5.00 g | E-2005/GR6 | 1.44 g | E-2005 |
| Purification No. 7 | 5.01 g | F-2005/GR7 | 2.25 g | F-2005 |

[(*) lyophilized]

### Example 7

### Compound (I/D)

### Examples of Analyses of Crude and Purified Fractions

Some lots of Compound (I/D) have been analyzed according to the following already described techniques:
Electrophoresis on polyacrylamide gel at 15%;
TNBS Test to determine the free amino groups;
Lowry Test to determine the molecular weight.

### 1-A) Electrophoresis on Polyacrylamide Gel at 15%

Compound (I/D) Crude (lots D-2005/GR5: E-2005/GR6: F-2005/GR7) Purified (lots D-2005; E-2005; F-2005), Fractions B (lots E-2005/GR6; F-2005/GR7) and rHLys.

By using the described technique, 5 mg of each sample under testing has been weighed, as indicated in Table n. 17. Each sample was then dissolved in 500 microlitres of bidistilled water and then applied on the plate.

TABLE n. 17
Tested samples and corresponding identification codes.

| Code | Sample | Source |
|---|---|---|
| 3GR | E-2005/GR6 | Synthesis n. 6 |
| 3A | E-2005 (fraction A) | Purification No. 6 |
| 3B | fraction B (E-2005/GR6) | Purification No. 6 |
| 4GR | D-2005/GR5 | Synthesis n. 5 |
| 4A | D-2005 (fraction A) | Purification No. 5 |
| 5GR | F-2005/GR7 | Synthesis n. 7 |
| 5A | F-2005 (fraction A) | Purification No. 7 |
| 5B | fraction B (F-2005/GR7) | Purification No. 7 |
| rHLys | Recombinant Human Lysozyme | — |

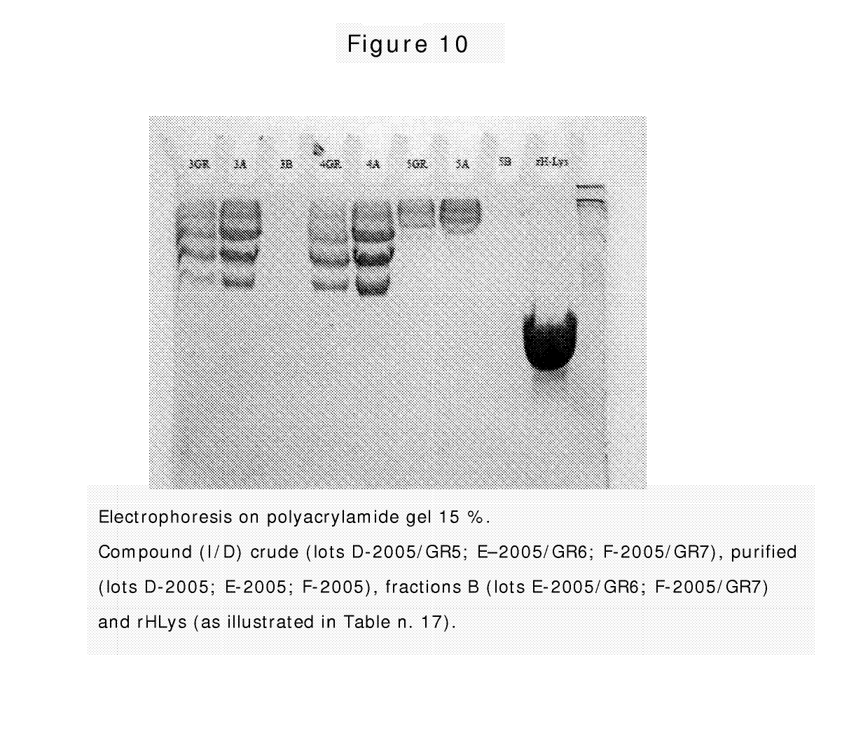

From the electrophoresis (FIG. 10) it is resulting that lots D-2005, E-2005 and F-2005, consisting of fraction A, are very rich of the more pegylated Compound (I/D), while the corresponding fractions B are consisting of partially pegylated products and of starting product rHLys.

### 2-A) TNBS Test—Lots of Purified Compound (I/D)

The products and the quantities reported in Table n. 18 were weighed and individually poured into 10.0 ml volumetric flasks. It has been then proceeded as previously directed.

TABLE n. 18
Products and weighed quantities with relevant
absorbance values at 421 nm.

| Products/lot | Weighed quantity | A 421 nm |
|---|---|---|
| Starting peptide (rHLys) | 5.0 mg | 0.623 |
| Compound (1/D) purified: | — | — |
| D-2005 (fraction A) | 5.1 mg | 0.162 |
| E-2005 (fraction A) | 5.0 mg | 0.226 |
| F-2005 (fraction A) | 5.0 mg | 0.056 |

The percentage of free amino groups (—NH₂) has been reported in Table n. 19.

TABLE n. 19
The percentage of free amino groups (—NH₂) in fractions A.

| Compound (I/D) purified/lot | % free amino groups |
|---|---|
| D-2005 (fraction A) | 6.37% |
| E-2005 (fraction A) | 9.07% |
| F-2005 (fraction A) | 2.25% |

### 3-A) Lowry Test—Lots of Purified Compound (I/D)

An example of Lowry Test is the analysis of the lots D-2005, E-2005 and F-2005 of the purified Compound (I/D). The test has been carried out by using the same techniques already described. After reading the values of the absorbance at 750 nm of the reference solutions and of the purified Compound (I/D) lots D-2005, E-2005 and F-2005, the calibration curve has been plotted (the concentration in the abscissa and the absorbance in the ordinate).

TABLE n. 19/A

| | C | A 750 nm |
|---|---|---|
| Reference 0 | 0 | 0 |
| Reference 1 | 1.23E−06 | 0.15 |
| Reference 2 | 3.08E−06 | 0.26 |
| Reference 3 | 4.94E−06 | 0.41 |

Calibration curve inherent to rHLys reference solutions is shown in FIG. 18.

After plotting the calibration curve, the value of ε was calculated, being in this case 84395, and by applying the equation: MW=g/l*ε/A the experimental value of the presumed molecular weight has been determined. The results of the considered lots were summarized in Table n. 20.

TABLE n. 20
Quantity of purified Compound (I/D) (fraction A) lots D-2005,
E-2005 and F-2005 in solution, relevant absorbances
and molecular weights.

| Product | | | |
|---|---|---|---|
| Compound (I/D) purified | Weight (g) | | |
| (fraction A) | in 5.0 ml | A 750 nm | MW (g/mole) |
| D-2005 (fraction A) | 4.08E−04 | 0.114 | 60705 |
| E-2005 (fraction A) | 4.08E−04 | 0.154 | 44937 |
| F-2005 (fraction A) | 4.08E−04 | 0.100 | 69203 |

ε=84395 A=ε*C C=g/l*MW

To determine the MW the following equation has been applied: MW=g/l*ε/A

### Conclusion of the Analytical Results

The tested lots of purified Compound (I/D) (fraction A) showed at the electrophoresis, at the TNBS Test and at the Lowry Test experimental results expected for the saturated conjugate, that is hexa-methoxypegylated-5 KDa, of the corresponding starting peptide rHLys.

### Example 8

### Syntheses of Compound (I/C) via mPEG-SMB 5 KDa

[mPEG-SMB 5 KDa=Activated reagent (IV)=methoxy polyethylene glycol succinimidyl alpha-methylbutanoate (mPEG=MW. about 5 KDa)]

### Synthesis n. 8 (Standard Conditions)

1.01 g of starting peptide (rHLys) were weighed, poured into a 250.0 ml volumetric flask and dissolved with 100.0 ml of pH 9.20 sodium tetraborate. 2.01 g of commercial mPEG-SMB 5 KDa were weighed and poured into the flask. The mixture was kept under constant stirring during 8 hours at 4° C. on an ice bath. Differently from the previous syntheses, after 2 hours the solution already presented a white precipitate. After 8 hours the solution was brought to pH 4.5 with 100.0% acetic acid. After this operation the solution still presented a residual precipitate. The solution was divided in two 250 ml volumetric flasks. The solutions in the flasks were cooled by means of the cold finger and lyophilized thus obtaining 6.01 g of crude Compound (I/C), lot G-2005/GR8.

### Synthesis n. 9 (Modified Standard Conditions)

1.01 g of starting peptide (rHLys) were weighed, poured into a 250.0 ml volumetric flask and dissolved with 50.0 ml of pH 9.20 sodium tetraborate. 2.01 g of commercially available mPEG-SMB 5 KDa were weighed in a second 250.0 ml flask and dissolved under constant stirring with 50.0 ml of pH 9.20 sodium tetraborate. The solution containing mPEG-SMB 5 KDa was then added to the solution of the first flask containing the starting peptide (rHLys). The mixture was kept under constant stirring during 8 hours at 4° C. on an ice bath. The solution was brought to pH 4.5 with 100.0% acetic acid. At the end of this operation the solution resulted completely clear. The solution was divided in two 250 ml volumetric flasks. The solutions were cooled by means of an ice finger and lyophilized obtaining 6.20 g of crude Compound (I/C), lot identified as H-2005/GR9.

### Synthesis n. 10 (Standard Conditions/Room Temperature)

1.01 g of starting peptide (rHLys) were weighed, poured into a 250.0 ml volumetric flask and dissolved with 100.0 ml of pH 9.20 sodium tetraborate. 2.01 g of commercially available mPEG-SMB 5 KDa was added. The mixture was kept under constant stirring during 8 hours at room temperature. After 2 hours the solution presented a whitish precipitate, as for previous Synthesis n. 8. After 8 hours the solution was brought to pH 4.5 with 100.0% acetic acid. At the end of this operation the solution still resulted not completely clear. The solution was divided in two 250 ml volumetric flasks. The solutions were cooled with iced finger and lyophilized thus obtaining 5.23 g of crude Compound (I/C), lot identified as I-2005/GR10.

### Synthesis n. 11 (Standard Conditions/Excess of Activated Reagent)

2.0 g of starting peptide (rHLys) were weighed, poured into a 250.0 ml volumetric flask and dissolved with 200.0 ml of pH 9.20 sodium tetraborate. 8.0 g of commercially available mPEG-SMB 5 KDa was added. The mixture was kept under constant stirring during 8 hours at a temperature of 4° C. on ice bath. After 2 hours the solution presented a whitish precipitate. After 8 hours the solution was brought to pH 4.5 with 100.0% acetic acid. After this operation the solution still resulted not completely clear, due to the presence of a precipitate, as in the case of previous Syntheses n. 8 and n. 10. The solution was divided in three 250 ml volumetric flasks, cooled and lyophilized thus obtaining 12.12 g of crude Compound (I/C), lot K-2005/GR11.

### Yields of the Syntheses of Crude Compound (I/C)

The yields of the 4 syntheses of crude Compound (I/C) have been summarized in Table n. 21.

TABLE n. 21
Yields of the syntheses of crude Compound (I/C).

| | | | Compound | |
|---|---|---|---|---|
| | | Used | (I/C) | |
| | | mPEG-SMB | crude + other | |
| Syntheses | rHLys | 5 KDa | fractions | Lot n. |
| Synthesis n. 8 | 1.01 g | 2.01 g | 6.01 g | G-2005/GR8 |
| (standard | | | | |
| conditions) | | | | |
| Synthesis n. 9 | 1.01 g | 2.01 g | 6.20 g | H-2005/GR9 |
| (modified | | | | |
| standard | | | | |
| conditions) | | | | |
| Synthesis n. 10 | 1.01 g | 2.01 g | 5.23 g | I-2005/GR10 |
| (standard | | | | |
| conditions/ | | | | |
| room | | | | |
| temperature) | | | | |
| Synthesis n. 11 | 2.00 g | 8.00 g | 12.12 g | K-2005/GR11 |
| (standard | | | | |
| conditions/ | | | | |
| reagent | | | | |
| in excess) | | | | |

### Example 9

Purification in column of the crude compound (I/C)

### Purification No. 8 of Lot G-2005/GR8

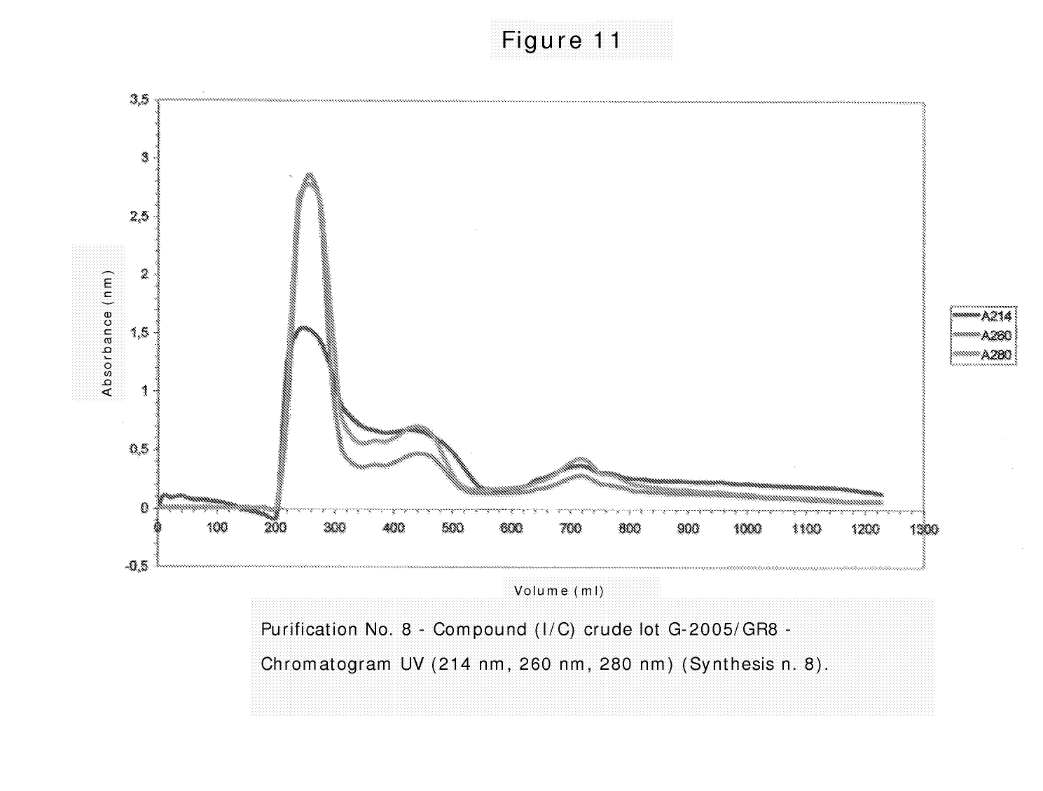

The same techniques and conditions already described for the purifications of Compound (I/A) have been adopted. 5.0 g of the crude Compound (I/C) lot G-2005/GR8 were dissolved with 24.0 ml of 0.2% acetic acid. The residual precipitate was withdrawn from the solution by centrifugation (4 times) at 4450 rpm at 4° C. The resulting whitish solution was loaded into the column, while the precipitate was collected on a Gooch filter (porosity 3), washed with ethyl ether and dried in a desiccator during 3 days. This highly insoluble precipitate seemed to be prevalently constituted from salts. The collected fractions, each of 20.0 ml, were analyzed at the UV light at three different wavelengths: 214 nm, 260 nm and 280 nm. On the basis of the obtained results, the recorded chromatogram (FIG. 11), attached as a reference for the subsequent purifications, and the obtained fractions (A, B and C) were grouped. The solution of fraction A, still presenting a slight opalescence, was divided in two aliquots, each of 100.0 ml, before lyophilization. The solutions of the fraction A were poured into two 250.0 ml volumetric flask, cooled and lyophilized. To lyophilize fraction B, the solution was equally divided in two aliquots of about 100.0 ml. Each aliquot was poured into a 250.0 ml volumetric flask, cooled and then lyophilized. Fraction C was also divided in two 250.0 ml flasks, cooled and lyophilized. 0.14 g of purified (fraction A) Compound (I/C) lot G-2005 were obtained, while 0.78 g were recovered from fraction B, containing conjugates with a different pegylation degree. The lyophilized fraction C (9 mg only) was withdrawn.

### Purification No. 9 of Lot H-2005/GR9

By using the same techniques and conditions already described for the previous purification, 5.0 g of crude Compound (I/C) lot H-2005/GR9 were dissolved with 24.0 ml of 0.2% acetic acid and loaded into a column. The collected fractions, each of 20.0 ml, were analyzed at the UV light at three different wavelengths and grouped on the basis of the recorded chromatogram: fraction A, containing Compound (I/C), and fraction B containing not reacted starting peptide. The solution containing fraction A were divided in 5 aliquots of 100.0 ml, poured into an identical number of 250.0 ml volumetric flasks, cooled and lyophilized. Also fraction B was submitted to the same operations. 0.62 g of purified (fraction A) Compound (I/C) lot H-2005 were obtained, while 1.28 g of fraction B (not reacted starting peptide rHLys) were recovered.

### Purification No. 10 of Lot I-2005/GR10

With the same techniques described herein above, 5.0 g of crude Compound (I/C) lot I-2005/GR10 were dissolved with 240 ml of 0.2% acetic acid, centrifugated 4 times, filtered on Gooch porosity 3, divided in fractions in a column, analyzed at the UV at three different wave lengths, and were grouped in two solutions on the basis of the recorded chromatogram. The solution fraction A, still presenting a slight opalescence, was divided in two aliquots of 100.0 ml each and poured into two 250.0 ml volumetric flask to be then cooled and lyophilized, and the solution fraction B was also submitted to the same procedure. 0.34 g of purified (fraction A) Compound (I/C), coded lot I-2005, were obtained, while 0.84 g of fraction B, consisting of product with a different pegylation degree, were recovered.

### Purification No. 11/a of Lot K-2005/GR11

By using the same above described techniques, 5.0 g of crude Compound (I/C) lot K-2005/GR11, dissolved with 24.0 ml of 0.2% acetic acid 0.2%, centrifugated 4 times, filtered on Gooch porosity 3, divided in fractions in a column, analyzed at the UV at three different wave lengths, and were grouped in two solutions on the basis of the recorded chromatogram. The solution fraction A, still presenting a slight opalescence, was divided in two aliquots of 100.0 ml each and poured into two 250.0 ml volumetric flask to be then cooled and lyophilized, and the solution fraction B was also submitted to the same procedure. 0.35 g of purified (fraction A) Compound (I/C) lot K-2005/1 were obtained, while 0.67 g of fraction B (product partially pegylated) were recovered.

### Purification No. 11/b of Lot K-2005/GR11

The above described purification process has been repeated on 5.01 g of crude Compound (I/C) lot K-2005/GR11. 0.27 g of purified (fraction A) Compound (I/C) lot K-2005/2 and also 0.73 g of fraction B (product partially pegylated) were obtained.

### Purification Yields of Crude Compound (I/C)

The purification yields of crude Compound (I/C) have been summarized in Table n. 22.

TABLE n. 22
Purifications of crude Compound (I/C)

| | | | Compound | |
|---|---|---|---|---|
| | Compound | | (I/C) | |
| | (I/C) | Lot n. | purified (*) | Lot n. |
| Purifications | crude | (crude) | (fraction A) | (purified) |
| Purification | 5.00 g | G-2005/GR8 | 0.14 g | G-2005 |
| No. 8 | | | | |
| Purification | 5.00 g | H-2005/GR9 | 0.62 g | H-2005 |
| No. 9 | | | | |
| Purification | 5.00 g | I-2005/GR10 | 0.34 g | I-2005 |
| No. 10 | | | | |
| Purification | 5.00 g | K-2005/GR11 | 0.35 g | K-2005/1 |
| No. 11/a | | | | |
| Purification | 5.01 g | K-2005/GR11 | 0.27 g | K-2005/2 |
| No. 11/b | | | | |

[(*) lyophilized]

### Example 10

### Compound (I/C)

### Examples of Analyses of Crude and Purified Fractions

Some lots of Compound (I/C) have been analyzed according to known methods and described techniques:
Electrophoresis on polyacrylamide gel at 15%;
TNBS Test to determine the free amino groups;
Lowry Test to determine the molecular weight.

### 1-A) Electrophoresis on Polyacrylamide Gel at 15%

Purified Compound (I/C) (Fraction A) Lot G-2005 and Fractions B and C

By using the described technique, 5 mg of each sample under testing has been weighed (table n. 23), dissolved in 500 microlitres of bidistilled water and then applied on the plate.

TABLE n. 23
Tested samples and corresponding identification codes.

| Code | Sample | Source |
|---|---|---|
| R | References of known molecular weight | — |
| | (16-20-30-40-50 KDa) | |
| rHLys | Recombinant Human Lysozyme | — |
| 9A | G-2005 (fraction A) | Purification No. 8 |
| 9B | fraction B (G-2005/GR8) | Purification No. 8 |
| 9C | fraction C (G-2005/GR8) | Purification No. 8 |

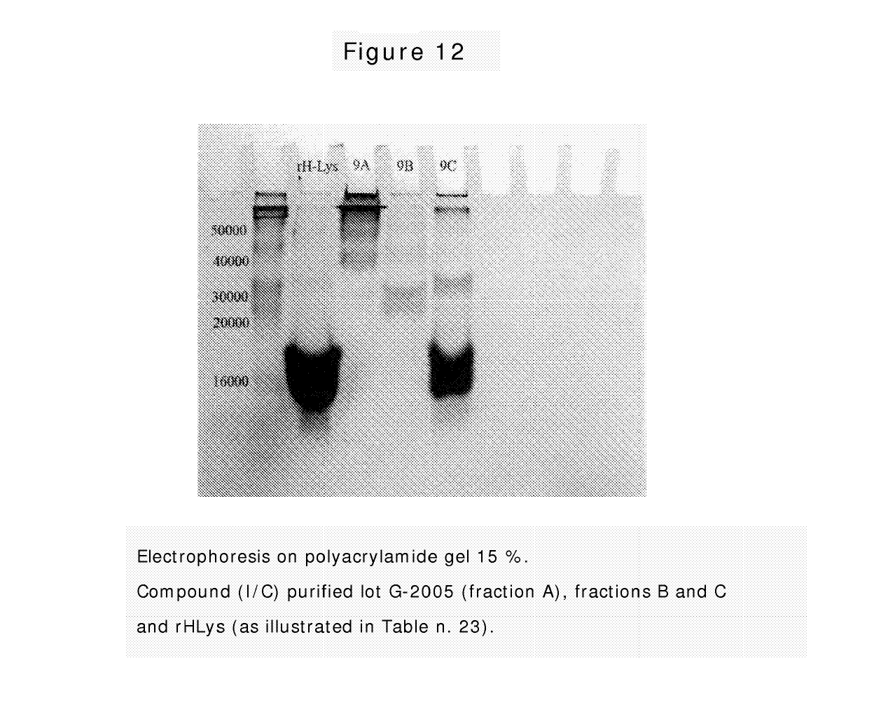

From the electrophoresis (FIG. 12) it has been observed that lot G-2005 (fraction A) is very rich of Compound (I/C), mostly pegylated, while fractions B and C are consisting of are of partially pegylated product and of starting product (rHLys).

### 1-B) Electrophoresis on Polyacrylamide Gel at 15%

Compound (I/C) Crude (Lots H-2005/GR9; I-2005/GR10) and Purified (lots H-2005; I-2005)

By using the described technique, 5 mg of each sample under testing has been weighed (Table n. 24); each sample was then dissolved separately in 500 microlitres of bidistilled water and then applied on the plate.

TABLE n. 24
Tested samples and corresponding identification codes.

| Code | Sample | Source |
|---|---|---|
| R | References of known molecular weight | — |
| | (16-25-45-50-60 KDa) | |
| rHLys | Recombinant Human Lysozyme | — |
| 10FR | H-2005/GR9 | Synthesis n. 9 |
| 10A | H-2005 (fraction A) | Purification No. 9 |
| 10B | fraction B (H-2005/GR9) | Purification No. 9 |
| 11GR | I-2005/GR10 | Synthesis n. 10 |
| 11A | I-2005 (fraction A) | Purification No. 10 |
| 11B | fraction B (I-2005/GR10) | Purification No. 10 |

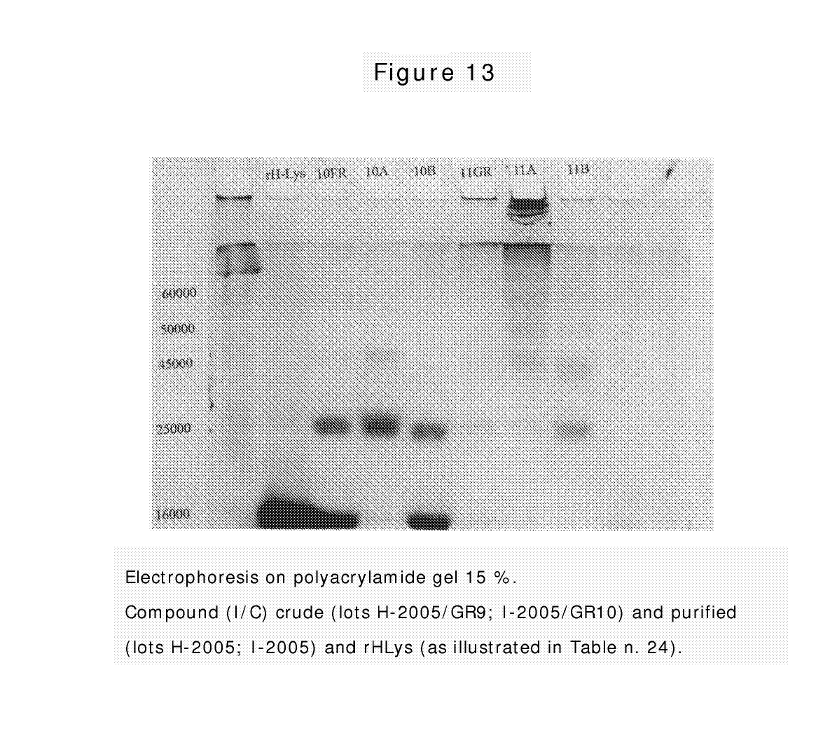

From the electrophoresis (FIG. 13) it has been observed that lot 1-2005 (fraction A) (Synthesis n. 10 and Purification No. 10) is the most rich of highly pegylated Compound (I/C), while are not much satisfactory the results of lot H-2005 (Synthesis n. 9), in which the starting product has scarcely reacted.

### 2-A) TNBS Test—Lots of Purified Compound (I/C)

The products and the quantities reported in Table n. 25 were weighed and individually poured into 10.0 ml volumetric flasks. It has been then proceeded as previously directed.

TABLE n. 25
Products and weighed quantities with relevant
absorbance values at 421 nm.

| Products/lot | Weighed quantity | A 421 nm |
|---|---|---|
| Starting peptide (rHLys) | 5.10 mg | 0.626 |
| Compound (1/D) purified: | — | — |
| G-2005 (fraction A) | 5.10 mg | 0.532 |
| H-2005 (fraction A) | 5.10 mg | 0.052 |
| I-2005 (fraction A) | 5.10 mg | 0.246 |

The percentage of free amino groups (—NH₂) has been reported in Table n. 26.

TABLE n. 26
The percentage of free amino groups (—NH₂) in fractions A.

| Purified Compound (I/C)/lot | % free amino groups |
|---|---|
| G-2005 (fraction A) | 21.24% |
| H-2005 (fraction A) | 2.08% |
| I-2005 (fraction A) | 19.84% |

### 3-A) Lowry Test—Lots of Purified Compound (I/C)

Lowry Test has been performed on the purified Compound (I/C) lots G-2005, H-2005 and I-2005. The test has been carried out by using the same techniques already described. After reading the values of the absorbance at 750 nm of the reference solutions and of the purified Compound (I/C) lots G-2005, H-2005 and I-2005, the calibration curve has been plotted (the concentration in the abscissa and the absorbance in the ordinate).

TABLE n. 26/A

| | C | A 750 nm |
|---|---|---|
| Reference 0 | 0 | 0 |
| Reference 1 | 1.23E−06 | 0.19 |
| Reference 2 | 3.08E−06 | 0.35 |
| Reference 3 | 4.94E−06 | |

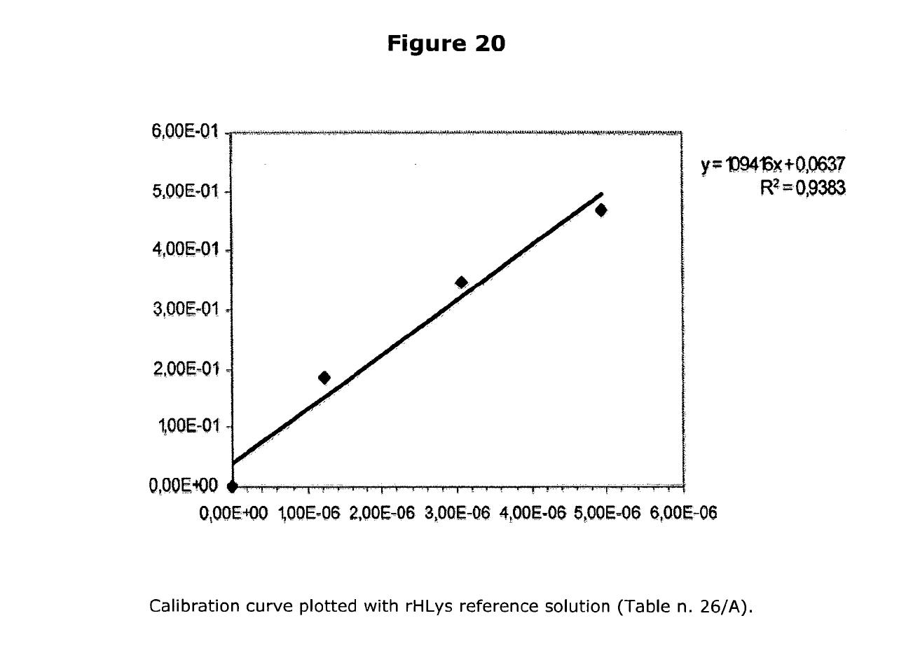

Calibration curve inherent to rHLys reference solutions is shown in FIG. 20.

After plotting the calibration curve, the value of ε was calculated, being in this case 109416, and by applying the equation: MW=g/l*ε/A the experimental value of the presumed molecular weight has been determined. The results of the considered lots were summarized in Table n. 27.

TABLE n. 27
Quantity of purified Compound (I/C) (fraction A) lots G-2005, H-2005
and I-2005 in solution, relevant absorbances and molecular weights.

| Product purified (fraction A) | Weigh (g) | | |
|---|---|---|---|
| Compound (I/C) | in 5.0 ml | A 750 nm | MW (g/mole) |
| G-2005 (fraction A) | 4.08E−04 | 0.297 | 30209 |
| H-2005 (fraction A) | 4.08E−04 | 0.156 | 57513 |
| I-2005 (fraction A) | 4.08E−04 | 0.321 | 27950 |

ε=109416 A=ε*C C=g/l*MW

To determine the MW the following equation has been applied: MW=g/l*ε/A

### Conclusion of the Previous Analytical Results

The tested lots of purified (fraction A) Compound (I/C) showed at the electrophoresis, at the TNBS Test and at the Lowry Test experimental results expected for the saturated conjugate, that is hexa-methoxypegylated-5 KDa, of the corresponding starting peptide rHLys.

### Example 11

### Synthesis of Compound (I/B) via mPEG-ButyrALD 6 KDa

[mPEG-ButyrALD 6 KDa=Activated reagent (III)=methoxy polyethylene-glycol butyraldehyde (mPEG-ButyrALD=MW about 6 KDa)]

### Synthesis n. 12 (Standard Conditions)

4.0 g of starting peptide rHLys were weighed, poured into a 250.0 ml metered flask and dissolved with 50.0 ml of sodium tetraborate pH 9.20. Thereafter, 2.01 g of commercial mPEG-ButyrALD 6 KD were weighed in a second 250.0 ml metered flask and were dissolved under constant stirring with 50.0 ml of sodium tetraborate pH 9.20. The solution containing mPEG-ButyrALD 6 Kda was then added to the solution of the first flask containing the starting peptide (rHLys). The mixture was then kept under constant stirring during 8 hours at 4° C. on ice bath. The solution was then brought to pH 4.5 with 100.0% acetic acid. At the end of this operation the solution resulted completely clear. The solution was equally divided in two 250.0 ml volumetric flasks. The solutions were cooled with an iced finger and lyophilized by obtaining 7.15 g of crude Compound (I/B) lot M-2005/GR12.

### Example 12

Purification in column of crude Compound (I/B).

### Purification No. 12/a of Lot M-2005/GR12

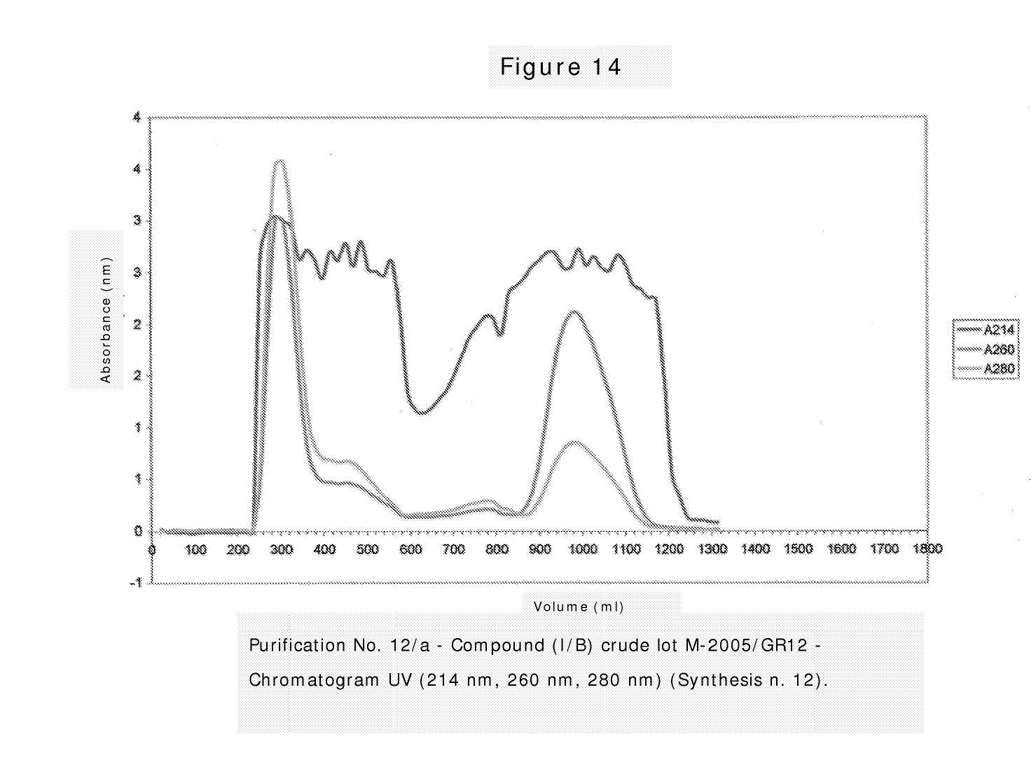

By using the same techniques and procedures already described for the purification of the other compounds, an aliquot of 5.0 g of crude Compound (I/B) lot M-2005/GR12, was dissolved with 24.0 ml of acetic acid 0.2%, centrifugated 4 times, filtered on Gouch porosity 3, separated by fractioning in column, analyzed at the UV light at three different wavelengths, were then regrouped in two solutions according to the recorded chromatogram (FIG. 14). The solution fraction A, still presenting a slight opalescence, was divided in two aliquots of 100.0 ml and poured in two 250.0 ml volumetric flasks, to be then cooled and lyophilized. The solution fraction B was submitted to the same treatment. 0.52 g of purified (fraction A) Compound (I/B) lot M-2005/1 were obtained, and 0.94 g of fraction B (partially pegylated product) were also recovered.

### Purification No. 12/b of Lot M-2005/GR12

The same purification process already described has been repeated on 2.01 g of crude Compound (I/B) lot M-2005/GR12. 0.17 g of purified (fraction A) Compound (I/B) lot M-2005/2 and also 0.51 g of fraction B (product partially pegylated) were obtained.

### Example 13

### Compound (I/B)

### Examples of Analyses of Crude and Purified Fractions

The Compound (I/B) has been analyzed according to the following methods and above described techniques:
Electrophoresis on polyacrylamide gel at 15%;
TNBS Test to determine the free amino groups;
Lowry Test to determine the molecular weight.

### 1-A) Electrophoresis on Polyacrylamide Gel at 15%

Purified Compound (I/B) Lots M-2005/1 and M-2005/2 (Fractions A and Fractions B)

By using the described technique, 5 mg of each sample under testing has been weighed (Table n. 28), then dissolved in 500 microlitres of bidistilled water and then applied on the plate.

TABLE n. 28
Tested samples and corresponding identification codes.

| Code | Sample | Source |
|---|---|---|
| R | References of known molecular | — |
| | weights (16-30-45-50 KDa) | |
| rHLys | Recombinant Human Lysozyme | — |
| 12 A 1° | M-2005/1 (fraction A) | Purification No. 12/a |
| 12 B 1° | fraction B (M-2005/GR12) | Purification No. 12/a |
| 12 A 2° | M-2005/2 (fraction A) | Purification No. 12/b |
| 12 B 2° | fraction B (M-2005/GR12) | Purification No. 12/b |

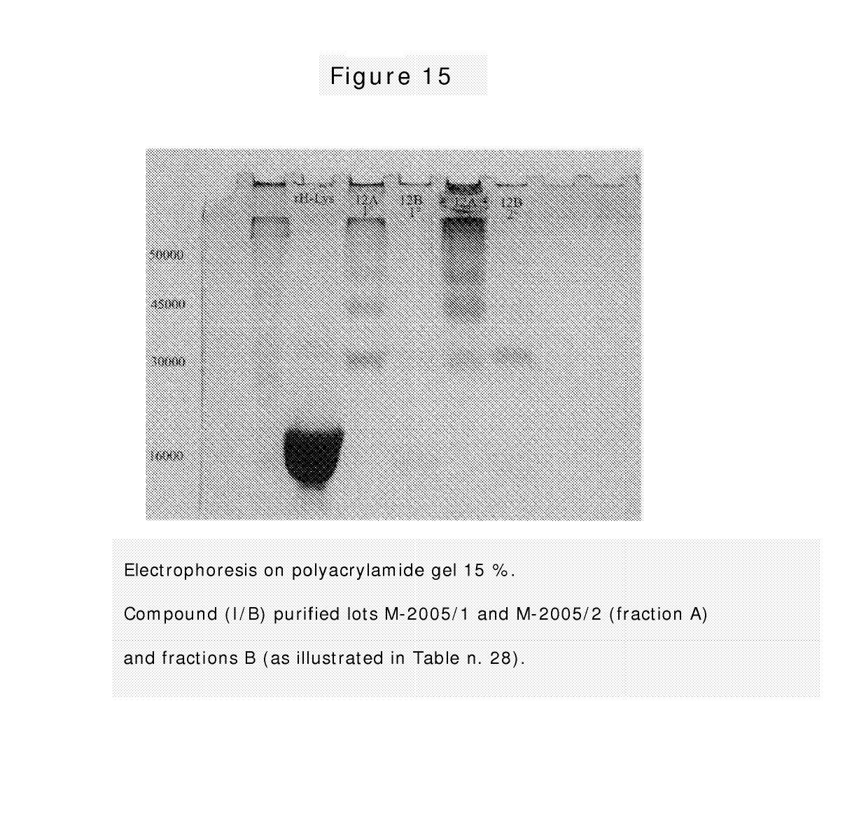

From the electrophoresis (FIG. 15) it has been observed that lot M-2005/2 (fraction A), resulting from Purification 12/b, is more reach of the higher pegylated Compound (I/B).

### 2-A) TNBS Test—Purified Compound (I/B)

The products and the quantities reported in Table n. 29 were weighed and individually poured into 10.0 ml volumetric flasks. It has been then proceeded as previously directed.

TABLE n. 29
Products and weighed quantities with relevant
absorbance values at 421 nm.

| Products/lot | Weighed quantity | A 421 nm |
|---|---|---|
| Starting peptide (rHLys) | 5.10 mg | 0.310 |
| Compound (1/B) purified | — | — |
| M-2005/1 (fraction A) | 5.10 mg | 0.061 |
| M-2005/2 (fraction A) | 5.10 mg | 0.030 |

The percentage of free amino groups (—NH₂) has been reported in Table n. 30.

TABLE n. 30
The percentage of free amino groups (—NH₂) in fractions A.

| Compound (I/B) purified/lot | % free amino groups |
|---|---|
| M-2005/1 (fraction A) | 4.92% |
| M-2005/2 (fraction A) | 2.42% |

### 3-A) Lowry Test—Purified Compound (I/C)

Lowry Test has been carried out by using the same techniques already described on the purified Compound (I/B) lots M-2005/1 and M-2005/2. After reading the values of the absorbance at 750 nm of the reference solutions and of the purified Compound (I/B) lots M-2005/1 and M-2005/2, the calibration curve has been plotted (the concentration in the abscissa and the absorbance in the ordinate).

TABLE n. 30/A

| | C | A 750 nm |
|---|---|---|
| Reference 0 | 0 | 0 |
| Reference 1 | 1.23E−06 | 0.19 |
| Reference 2 | 3.08E−06 | 0.35 |
| Reference 3 | 4.94E−06 | 0.47 |

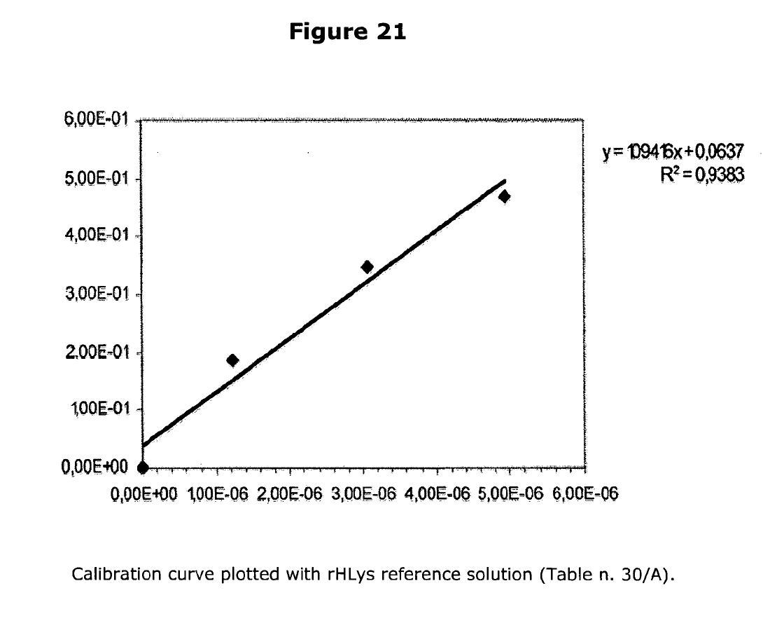

Calibration curve inherent to rHLys reference solutions is drawn in FIG. 21. After plotting the calibration curve, the value of ε was calculated, being in this case 109416, and by applying the equation: MW=g/l*ε/A the experimental value of the presumed molecular weight has been determined. The results of the considered lots were summarized in the following Table n. 31.

TABLE n. 31
Quantity of purified Compound (I/B) (fraction A) lots M-2005/1 and
M-2005/2 in solution, relevant absorbances and molecular weights.

| Product Compound (I/B) | | | |
|---|---|---|---|
| purified | Peso (g) | | |
| (fraction A) | in 5.0 ml | A 750 nm | MW (g/mole) |
| M-2005/1 (fraction A) | 4.08E−04 | 0.226 | 39700 |
| M-2005/2 (fraction A) | 4.08E−04 | 0.148 | 60418 |

ε=109416 A=ε*C C=g/l*MW

To determine the MW the following equation has been applied: MW=g/l*ε/A

### Conclusion of the Previous Analytical Results

Particularly lot M-2005/2 of purified Compound (I/B) (fraction A) showed at the electrophoresis, at the TNBS Test and at the Lowry Test experimental results expected for the saturated conjugate (hexa-methoxypegylated-6 Kda) of the corresponding starting peptide.

### Example 14

### Effects on Tumour Growth and Metastases Development of Experimental Carcinoma of Orally Administered Compound (I/A) Lot A-2004 in Mouse

### a) State of the Art

A number of scientific evidences supporting the modulation of the immuno response of GALT (Gut Associated Lymphoid Tissues) and also Applicant's hypothesis involving cytokyne-related immunological mechanisms of activation of some recently discovered receptors, such as the Toll-Like Receptors (TLR's) of lymphocytes (Li L., Inflammation & Allergy, 3, 81-4, 2003; Vogel S. N. et al., Molecular Interventions, 3, 466-77, 2003), may represent a suitable system to treat abnormal cell proliferation and related systemic pathologies.

### b) Aim of the Study

The purpose of the present research study was aiming to compare in vivo the effects of Compound (I/A) Lot A-2004 of the invention on the tumour growth and metastatic development in mouse model of experimental carcinoma. Compound (I/A) Lot A-2004 was administered orally for 14 consecutive days, admixed to the powdered food. The effect of Compound (I/A) Lot A-2004 was compared to that of commercially available Hen Egg-white Lysozyme (HEL).

### c) Drugs and Dose Levels

The Compound (I/A) Lot A-2004 used for the experimental study was synthesized starting from high quality (purity>90.0%) starting peptide rHLys and then purified according to the methods of the previous Examples of the instant invention. HEL, extracted from hen egg-white, used as a reference product for the research, was currently available on the market (SPA Societá Prodotti Antibiotici S. p. A., Milano, Italy).

Both the synthesis and purification of Compound (I/A) Lot A-2004 and the research study in mouse were performed on behalf and in accordance to Applicant's instructions by external and independent Contract Research Organizations (CRO's).

Compound (I/A) Lot A-2004 was administered at three dose levels of 25 mg/kg/day, 50 mg/kg/day and 100 mg/kg/day, while HEL was administered at the higher dose level of 100 mg/kg/day. All compounds were added to the daily powdered food for 14 consecutive days. Food consumption was calculated separately and the medicated food was renewed daily. The amount of food discarded by the animals influenced the daily dosage by less than 10.0%.

### d) Animal Model

CBA female mice, of 17-18 g of body weight, used for the test were sourced by HARLAN Italy Srl, S. Pietro al Natisone (UD), Italy. The animals were allowed to be fed with powdered food for 7 days before the experiment. The animals were maintained under conventional procedures (with drinkable tape-water ad libitum) in suitable Animal House facilities at the Department of Biomedical Sciences of the University of Trieste, which were partially hired by the appointed CRO.

### e) Solid Tumour Model

The murine mammary carcinoma line (MCa) used in the present work derived from a spontaneous solid tumour of CBA female mice, subsequently isolated and stabilised at Rudjer Boskovic Institute, Zagreb, Croatia. The tumour cell line in use at Callerio Foundation Onlus, Trieste, Italy was stored in cryogenic tanks with liquid nitrogen, in cryogenic vials containing 107 viable cells of a single-cell suspension obtained from primary tumours at the third transplant generation. The tumour was grown in vivo by implanting intramuscularly (i.m.) into the calf of the left hind leg of CBA female mice the content of one vial of cells of MCa taken from the cryogenic tank. The tumour of two mice was harvested from the legs after 14 days (about 2 g of tumour mass) and a single-cell suspension was prepared and re-implanted into 4 CBA female mice which constitute the donors for tumour propagation for the experimental purposes. The tumour, harvested from mice under sterile conditions (animals were killed by cervical dislocation, the area of tumour growth was disinfected with an appropriate solution and the animals were placed under a sterile box), were pooled, minced with scissors, diluted with Dulbecco's phosphate buffered saline (pH 7.4) containing Ca⁺² and Mg⁺² (PBS) and filtered with a double layer of sterile gauze to remove tissue debris. The cell suspension was then centrifuged at 250×g at 0-4° C. for 10 min; the supernatant was discarded while the pellet was re-suspended in an equal volume of PBS, and cell concentration and viability was determined by the trypan blue exclusion test with a Burker's camera. Viable cells were finally diluted in order to have 1.0×10⁶ cells/0.05 ml PBS.

For the experimental purposes, 110 CBA mice were implanted i.m. into the calf of the left hind leg with 106viable MCa carcinoma cells of a cell suspension obtained from the primary tumour harvested from the 4 CBA donors.

### f) Experimental Protocol

Primary tumours were implanted i.m. on Day 0 (Feb. 3, 2005). Mice were then randomized into the test groups (Table 17). The protocol was constituted of four arms of treatments and one arm of control.

Primary tumour growth was evaluated at Day 7 (Feb. 10, 2005), on Day 9 (Feb. 12, 2005), on Day 11 (Feb. 14, 2005), on Day 13 (Feb. 16, 2005), on Day 15 (Feb. 18, 2005) and on Day 18 (Feb. 21, 2005). Measurements of the two orthogonal axis of the tumour were done by means of a calliper. Primary tumour weight was calculated by the formula: (π/6)×a²×b, where a and b are the minor and major perpendicular axis respectively and subtracting to the result the weight of the tumour-free leg hind (that correspond to about 100 mg). Lung metastases number and weight were evaluated on Day 20 (Feb. 23, 2005) after killing the animals by cervical dislocation. Lung metastases were counted and measured on Day 20 (Feb. 23, 2005) by means of a low-power stereo microscope equipped with a graduated grid. Metastases' weight was determined by the formula: (π/6)×a²×b, where a and b are the minor and major orthogonal axis respectively.

Spleens weight of three random chosen animal per group was taken on Day 20 (Feb. 23, 2005) after killing the animals by cervical dislocation.

Cages containing animals were controlled daily to prevent suffering of tumour-bearing mice.

TABLE 32
Design of the treatment arms of experimental protocol.

| | Compound (I/A) |
|---|---|
| | Lot A-2004 |
| Groups | mg/kg/day |
| Controls | — |
| HEL 100 | 100 |
| Compound (I/A) 100 = C. (I/A) 100 | 100 |
| Compound (I/A) 50 = C. (I/A) 50 | 50 |
| Compound (I/A) 25 = C. (I/A) 25 | 25 |

Compound (I/A) Lot A-2004 = C. (I/A)

### g) Tabulation and Results

The determined results of the considered parameters are summarized in the following Tables from 33 to 37.

TABLE 33
Animal deaths during treatment (Day 0-Day 20).

| | Feb. | Feb. | Feb. | Feb. | Feb. | Feb. | Feb. | |
|---|---|---|---|---|---|---|---|---|
| | 3^{rd} | 18^{th} | 19^{th} | 20^{th} | 21^{st} | 22^{nd} | 23^{rd} | |
| | 2005 | 2005 | 2005 | 2005 | 2005 | 2005 | 2005 | |
| | Progressive day of treatment | | | | | | | Total |
| Groups | 0 | 15 | 16 | 17 | 18 | 19 | 20 | deaths |
| Controls | 0/10 | 1/10 | 0/9 | 0/9 | 0/9 | 1/9 | 1/8 | 3/10 |
| HEL 100 | 0/9 | 1/9 | 1/8 | 0/7 | 1/7 | 1/6 | 1/5 | 5/9 |
| C. (I/A) 100 | 0/9 | 0/9 | 0/9 | 0/9 | 0/9 | 0/9 | 1/8 | 1/8 |
| C. (I/A) 50 | 0/8 | 0/8 | 0/8 | 0/8 | 0/8 | 0/8 | 0/8 | 0/8 |
| C. (I/A) 25 | 0/8 | 0/8 | 0/8 | 0/8 | 0/8 | 0/8 | 1/8 | 1/8 |

Compound (I/A) Lot A-2004 = C. (I/A)

The number of living animals per each group was indicated, starting from the day of tumour implant (Day 0) up to the day of sacrifice (Day 20). The total number of animals that died before the end of the experiment was 10/43.

TABLE 34
Body weight variations (in g) between Day 0 (day of tumour implant)
and Day 20 (end of the experiment and metastases evaluation).

| Groups | Body weight variation (in g) during treatment period | | |
|---|---|---|---|
| Product and | Day 0 | Day 20 | Variation (% of the |
| daily dose | mean ± S.E. | mean ± S.E. | respective Controls) |
| Controls | 17.6 ± 0.3 | 18.3 ± 0.3 | — |
| HEL 100 | 18.3 ± 0.4 | 17.5 ± 0.7 | −8.0 vs Controls |
| C. (I/A) 100 | 17.8 ± 0.3 | 18.6 ± 0.5 | 0.5 vs Controls |
| C. (I/A) 50 | 17.9 ± 0.2 | 18.4 ± 0.4 | −1.1 vs Controls |
| C. (I/A) 25 | 18.2 ± 0.4 | 19.0 ± 0.5 | 0.4 vs Controls |

Compound (I/A) Lot A-2004 = C. (I/A)

Weight variations were calculated according to the formula

{[(Group X m.w. on Day 20/Group X m.w. on Day 0)/(Group Y m.w. on Day 20/Group Y m.w. on Day 0)]*100}−100

m.w=mean weight

TABLE 35
Effect on primary tumour growth (in mg).

| | Primary tumour mass (mg) - mean ± S.E. | | | | | |
|---|---|---|---|---|---|---|
| | Date of treatment | | | | | |
| | Feb. 10^{th} | Feb. 12^{th} | Feb. 14^{th} | Feb. 16^{th} | Feb. 18^{th} | Feb. 21^{st} |
| | 2005 | 2005 | 2005 | 2005 | 2005 | 2005 |
| | Progressive day of treatment | | | | | |
| Groups | 7 | 9 | 11 | 13 | 15 | 18 |
| Controls | 246 ± 15 | 350 ± 15 | 642 ± 43 | 1043 ± 63 | 1519 ± 85 | 2682 ± 126 |
| HEL 100 | 231 ± 24 | 338 ± 21 | 591 ± 35 | 1062 ± 104 | 1325 ± 189 | 2133 ± 130 |
| C. (I/A) 100 | 241 ± 22 | 338 ± 21 | 559 ± 35 | 1000 ± 48 | 1504 ± 62 | 2173 ± 90 |
| C. (I/A) 50 | 233 ± 24 | 370 ± 29 | 556 ± 52 | 881 ± 109 | 1432 ± 116 | 2022 ± 165 |
| C. (I/A) 25 | 214 ± 24 | 336 ± 24 | 563 ± 75 | 1074 ± 130 | 1292 ± 99 | 1930 ± 111 |

Compound (I/A) Lot A-2004 = C. (I/A)

TABLE 36
Effect on lung metastasis formation.

| | Lung metastases | | |
|---|---|---|---|
| | | Weight | |
| | Number/mouse | (mg/mouse) | Metastases- |
| Groups | mean ± S.E. | mean ± S.E. | free animals |
| Controls | 38.0 ± 3.0 | 69.86 ± 14.75 | 2/7 |
| HEL 100 | 31.0 ± 8.0 | 41.76 ± 28.69 | 0/4 |
| C. (I/A) 100 | 22.0 ± 3.0 | 11.50 ± 6.13 | 0/8 |
| C. (I/A) 50 | 23.0 ± 5.0 | 16.71 ± 6.78 | 0/8 |
| C. (I/A) 25 | 14.0 ± 4.0 | 3.25 ± 1.26 | 0/7 |

Compound (I/A) Lot A-2004 = C. (I/A)

TABLE 37
Spleen weights (in g) at the day of sacrifice (Day 20).

| | Spleen weight | % of body |
|---|---|---|
| Groups | mean ± S.E. | weight (*100) |
| Controls | 0.28 ± 0.01 | 1.55 |
| HEL 100 | 0.34 ± 0.07 | 1.94 |
| C. (I/A) 100 | 0.29 ± 0.03 | 1.56 |
| C. (I/A) 50 | 0.35 ± 0.03 | 1.92 |
| C. (I/A) 25 | 0.31 ± 0.02 | 1.63 |

Compound (I/A) Lot A-2004 = C. (I/A)

### h) Result Summary and Conclusions

### h.1) Effects on Primary Tumour Growth

h. 1.1) Results Summary
Compound (I/A) Lot A-2004, at equimolecular doses corresponding to 100 mg, 50 mg and 25 mg/kg/day of starting peptide (rHLys), significantly reduced the growth of primary tumour (p<0.01 versus untreated controls) on Day 18^{th} from tumour implant.
Compound (I/A) Lot A-2004 showed at the administered doses also a certain antitumour effect on MCa mammary carcinoma at the primary tumour site.
HEL (100 mg/kg/day) was slightly effective on primary tumour growth (p<0.05 versus untreated controls).
Compound (I/A) Lot A-2004 at each of the different dose levels (100 mg, 50 mg and 25 mg/Kg/day) was significantly more effective (p<0.01 versus untreated controls) on the reduction of primary tumour growth than 100 mg/Kg/day of HEL (p<0.05 versus untreated controls).

### h.2) Effects on Metastases Development

h.2.1) Results Summary
All dose levels of Compound (I/A) Lot A-2004, at equimolecular doses corresponding to 100 mg, 50 mg and 25 mg/kg/day of starting peptide (rHLys), significantly reduced the mean overall lung metastases mass (p<0.01 versus untreated controls, but also the mean metastases number (p<0.05-p<0.01 versus untreated controls). The reduction of metastases mass and number did not appear to be dose related and this observation shall be focused on further researches.

HEL, at 100 mg/Kg/day, was ineffective on both metastases growth and number.

### h.3) Conclusions

Compound (I/A) Lot A-2004, at equimolecular doses corresponding to 100 mg, 50 mg and 25 mg/kg/day of starting peptide (rHLys), significantly reduced the overall lung metastases mass and showed to be remarkably effective on metastases number.
The effectiveness of Compound (I/A) Lot A-2004 on metastasis development was independent from the administered dose, at least in the used range of doses from 25 mg to 100 mg/Kg/day.
Compound (I/A) Lot A-2004 showed also a certain antitumour effect on MCa mammary carcinoma at the primary tumour site.

Although the foregoing invention has been described in some detail by way of illustration and examples, for purposes of clarity of understanding, it will be obvious that certain changes and modifications may be practised by a normal skilled artisan within the scope of the appended claims.

1. A compound having a general formula (I): 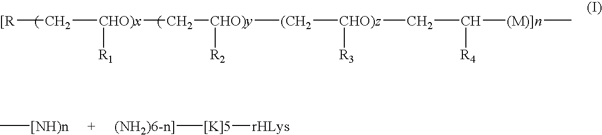
wherein:
R is a lower alkoxy, where the term lower alkoxy designates a linear or ramified alkoxy from 1 through 6 carbon atoms;
R₁, R₂, and R₃, independently from each other, are selected from a hydrogen atom or a lower alkyl group, where the term lower alkyl designates a linear or ramified alkyl from 1 through 6 carbon atoms;
R₄ is a hydrogen atom or a lower alkyl group, provided that R₄ is absent when (M) is a hydrogen atom;
x, y and z are selected from any combination of numbers such that the resulting molecular weight (MW) of any of the selected polyethylene glycol moieties vary from 300 to 66,000 Daltons (from 0.3 to 66 Kilodaltons, i.e. from 0.3 KDa to 66 KDa);
M is a linker group;
“n” represents an integer from 1 to 6, being the number of polyethylene glycols moieties linking one or more out of the six free amino groups of the five lysines present in rHLys (recombinant human lysozyme);
[(NH)n+(NH₂)6−n] are the six amino groups of the five lysines present in rHLys, where (NH)n represents one or more amino groups linked to “n” polyethylene glycol moieties and (NH₂6−n represents one or more residual free amino groups;
[K]5 are the five lysines (K) present in rHLys chain; and
[(NH)n+(NH₂)6−n]-[K]5-rHLys is a detailed representation of recombinant human lysozyme, where the five lysines [K]5 present in recombinant human lysozyme and the six linked or free amino groups [(NH)n+(NH₂)6−n] are drawn separately.

2. The compound according to claim 1 wherein:
the linker group M is derived from one or more compounds that react with a linear or branched polyethylene glycol to produce an activated linear or branched polyethylene glycol, which in turn will react and substitute one hydrogen of one or more free amino groups of the lysines in the recombinant human lysozyme to thereby link together the polyethylene glycol moiety and the recombinant human lysozyme.

3. The compound of claim 2, wherein the linker group M is selected from at least one of (a), (b), (c) or (d), each originating a different type of linkage, where:
(a) is a hydrogen atom, provided that R₄ is absent;
(b) is a methylene group;
(c) is

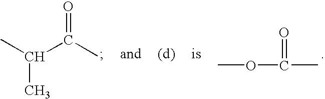

4. The compound according to claim 3, wherein:
the recombinant human lysozyme moiety of the compound, represented in the formula (I) as —[(NH)n+(NH₂)6−n]-[K]5-rHLys, is obtained either by a DNA recombinant technique for producing proteins in plant seeds, such as maturing rice grains transformed using codon-optimized structural gene for human lysozyme, or by a fermentation technique where a selected microorganism has been genetically modified with a gene construct to yield human lysozyme.

5. The compound according to claim 4, wherein the polyethylene glycol moiety of the compound presents preferably:
R that is a methoxy group; and
R₁, R₂, and R₃ that are simultaneously a hydrogen atom;
provided that R4 is absent when the linker M is a hydrogen atom.

6. (canceled)

7. The compound according to claim 1, wherein:
the polyethylene glycol moiety of the compound presents a molecular weight variable from 400 to 10,000 Daltons.

8. (canceled)

9. The compound of claim 5, consisting of:
a mono-, di-, tri-, tetra-, penta-, or a hexa-pegylated conjugate of recombinant human lysozyme.

10. The compound according to claim 9 consisting of a hexa-pegylated conjugate of recombinant human lysozyme.

11. A method to produce a compound according to claim 1, comprising the step of:
reacting chemically a recombinant human lysozyme with at least one activated polyethylene glycol selected from the group consisting of:
(a) TalkoxyPEG (alkoxypolyethylene glycol tresylate);
(b) alkoxyPEG-ButyrALD (alkoxypolyethylene glycol butyraldehyde);
(c) alkoxyPEG-SMB (alkoxypolyethylene glycol succinimidyl alfa-methylbutanoate); and
(d) alkoxyPEG-OSu (also known as alkoxyPEG-SC) (alkoxypolyethylene glycol succinimidyl carbonate).

12. A method to produce a compound according to claim 1, comprising the step of:
reacting chemically a recombinant human lysozyme with at least one activated polyethylene glycol is selected from the group consisting of:
(a) TmPEG (methoxypolyethylene glycol tresylate);
(b) mPEG-ButyrALD (methoxypolyetylene glycol butyraldehyde);
(c) mPEG-SMB (methoxypolyethylene glycol succinimidyl alfa-methylbutanoate); and
(d) mPEG-OSu (also known as methoxyPEG-SC) (methoxypolyethylene glycol succinimidyl carbonate).

13. The method according to claim 12, wherein:
the reacting step is achieved with an excess of the selected activated polyethylene glycol relative to the recombinant human lysozyme at a reaction temperature not exceeding 10° C.

14. Use of a compound according to claim 1 in the preparation of a pharmaceutical composition for the treatment of a disease associated with abnormal cell proliferation.

15. A pharmaceutical composition comprising:
at least one compound according to claim 1; and
a pharmaceutically acceptable vehicle or support.

16. A pharmaceutical composition according to claim 15 for the treatment of a disease associated with abnormal cell proliferation.

17. The compound of claim 1, wherein:
the linker group M is selected from at least one of (a), (b), (c) or (d), each originating a different type of linkage, where:
(a) is a hydrogen atom, provided that R₄ is absent;
(b) is a methylene group;
(c) is

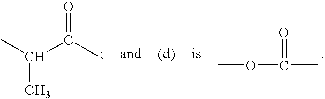

18. The compound of claim 1, wherein:
the recombinant human lysozyme moiety of the compound, represented in the formula (I) as —[(NH)n+(NH₂)6−n]-[K]₅-rHLys, is obtained either by a DNA recombinant technique for producing proteins in plant seeds, such as maturing rice grains transformed using codon-optimized structural gene for human lysozyme, or by a fermentation technique where a selected microorganism has been genetically modified with a gene construct to yield human lysozyme.

19. The compound according to claim 1, wherein:
the polyethylene glycol moiety of the compound presents preferably:
R that is a methoxy group; and
R₁, R₂, and R₃ that are simultaneously a hydrogen atom;
provided that R₄ is absent when the linker M is a hydrogen atom.

20. The compound of claim 1, consisting of:
a mono-, di-, tri-, tetra-, penta-, or a hexa-pegylated conjugate of recombinant human lysozyme.

21. The method according to claim 11, wherein:
the reacting step is achieved using an excess of the selected activated polyethylene glycol relative to the recombinant human lysozyme at a reaction temperature not exceeding 10° C.
